(19) United States
(12) Patent Application Publication
(10) Pub. No.: US 2018/0104317 A1
(43) Pub. Date: Apr. 19, 2018

(54) METHODS AND PHARMACEUTICAL COMPOSITIONS FOR TREATING CELIAC DISEASE AND GLUTEN INTOLERANCE

(71) Applicant: Immunogenics LLC, Newport Beach, CA (US)

(72) Inventor: Daniel C. Adelman, Redwood City, CA (US)

(21) Appl. No.: 15/833,536

(22) Filed: Dec. 6, 2017

Related U.S. Application Data
(63) Continuation of application No. 14/234,531, filed on Mar. 11, 2014.
(60) Provisional application No. 61/550,729, filed on Oct. 24, 2011.
(60) Provisional application No. 61/511,401, filed on Jul. 25, 2011.

(51) Int. Cl. A61K 38/54; A23L 33/17; A23L 2/66; A61K 9/20; A61K 38/48; A61K 9/19; A61K 9/00

(52) U.S. Cl. CPC A61K 38/54; A23L 33/17; A23L 2/66; A61K 9/2054; A61K 9/2018; A61K 9/2013; A61K 9/2009; A61K 9/19; A61K 9/0095; A61K 38/48

(57) ABSTRACT

The invention concerns methods for protecting a subject in need from a deleterious effect of gluten ingestion. The invention specifically concerns the treatment of celiac disease and gluten intolerance. The invention further provides pharmaceutical compositions for protecting a subject in need from a deleterious effect of gluten ingestion, and, in particular, for treating celiac disease and gluten intolerance. Oral administration of ALV003 can protect celiac disease patients and patients otherwise suffering from gluten-intolerance from the harmful effects of ingesting food containing gluten.

### CROSS REFERENCE

This application claims benefit and is a Continuation of application Ser. No. 14/234,531 filed Mar. 11, 2014, which is a 371 application and claims the benefit of PCT Application No. PCT/US2012/048149, filed Jul. 25, 2012, which claims benefit of U.S. Provisional Patent Application Nos. 61/550,729, filed Oct. 24, 2011 and 61/511,401, filed Jul. 25, 2011, which applications are incorporated herein by reference in their entirety.

### FIELD OF THE INVENTION

The invention concerns methods for protecting a subject in need from a deleterious effect of gluten ingestion. The invention specifically concerns the treatment of celiac disease and gluten intolerance. The invention further provides pharmaceutical compositions for protecting a subject in need from a deleterious effect of gluten ingestion, and, in particular, for treating celiac disease and gluten intolerance.

### BACKGROUND OF THE INVENTION

### Celiac Disease

Celiac disease is an acquired chronic immune disorder that develops in susceptible individuals (many of whom are of HLA genotype DQ2 or DQ8) related to an environmental factor, gluten, which is the storage protein of wheat and related grains like rye and barley [(Fasano and Catassi 2001), (Van Heel and West 2006), (Rostom, Murray et al. 2006), (Green and Cellier 2007); a list of the full citation of references cited herein by author name(s) and year of publication is provided at the end of the detailed description of the invention]. The prevalence of celiac disease in Europe and in the United States has been estimated to be approximately 1-2% of the population [(Mäki, Mustalahti et al. 2003), (Fasano, Berti et al. 2003), (West 2003), (Bingley, Williams et al. 2004), (Shan, Qiao et al. 2005), (Van Heel and West 2006), (Green 2007)]. Celiac disease has a wide range of clinical manifestations including latent or silent celiac disease, disease with only mild gastrointestinal disturbances, chronic gastrointestinal symptoms, malabsorption, and/or weight loss. Celiac disease is often diagnosed in patients with isolated iron deficiency anemia.

The ingestion of gluten-containing cereals can also induce manifestations outside the gut (Mäki and Collin 1997), such as osteoporosis, peripheral and central nervous system involvement (Hadjivassiliou 2006), mild or severe liver disease, infertility problems, and the classical example is the gluten-induced skin disease, dermatitis herpetiformis. Dermatitis herpetiformis (DH) is a cutaneous manifestation of celiac disease in which an intensely pruritic, herptiform rash can present on the elbows, knees, buttocks, and scalp of a celiac disease patient in response to ingestion of gluten. The rash is characterized by high IgA deposits seen histologically in the upper papillary dermis. The symptoms and histology of the rash improve with adherence to a gluten free diet. Approximately 10% of patients diagnosed with celiac disease will manifest DH. The gluten-induced small bowel pathology in celiac disease is characterized by an inflammatory reaction that is accompanied by villus atrophy and hypertrophy of crypts (Kagnoff 2007).

The only accepted gold standard for celiac disease diagnosis is the finding of gluten-induced small intestinal mucosal injury [(Walker-Smith 1990), (Rostom, Murray et al. 2006)]. Clinical findings are usually equivocal: newly diagnosed patients eating normal gluten-containing food may be totally symptomless or have only vague gastrointestinal symptoms whereas in others symptoms may be severe; in people with extra-intestinal manifestations gastrointestinal symptoms may also be absent, thus having a clinically silent celiac disease. One feature that is common to all however is the manifest gluten-sensitive small intestinal mucosal lesion. In untreated celiac disease, the length of functionally impaired bowel determines the degree of malabsorption and the presence of symptoms does not relate at all to the histological features of the proximal biopsy [(Macdonald, Brandborg et al. 1964), (Marsh and Crowe 1995)]. This also explains why oral glucose tolerance tests, fecal fat excretion, d-xylose excretion tests, hematologic investigations, and radiologic examination of the small bowel fail to distinguish patients with suspected malabsorption from those with or without mucosal atrophy and, thus, frequently give misleading results (Sanderson 1975). Only patients with extensive and severe enteropathy will have evidence of steatorrhea and increased intestinal permeability; in patients with mild-to-moderate enteropathy these tests may remain normal, and therefore these tests are no longer important tools in cases of suspected celiac disease or while monitoring dietary treatment (Farrell and Kelly 2002). Furthermore, recent guidelines and management models for celiac disease diagnosis and treatment in the USA no longer recommend these functional studies [(Hill I D 2005), (Rostom, Murray et al. 2006)]. Instead, during the last two decades, highly sensitive and specific gluten-dependent serum autoantibody tests have been used for celiac disease case finding, population-based screening studies, monitoring the gluten-free diet, and measurement of mucosal relapse on gluten challenge [(Mäki, Hallstrom et al. 1984), (Mäki, Landeaho et al. 1989), (Mäki 1991), (Mäki 1995), (Dieterich 1998), (Sulkanen, Halttunen et al. 1998), (Mustalahti 2002), (Kaukinen, Halme et al. 2002), (Mäki, Mustalahti et al. 2003), (Korponay-Szabo, Raivio et al. 2005), (Collin 2005), (Hill I D 2005), (Holm, Mäki et al. 2006), (Raivio 2006), (Rostom, Murray et al. 2006), (Kurppa 2009)].

For patients with celiac disease, lifelong complete gluten exclusion needs to be strictly followed to avoid a substantially enhanced risk for the development of further complications, such as bone disorders, infertility, and cancer [(Peters, Askling et al. 2003), (Van Heel and West 2006), (Rostom, Murray et al. 2006), (Green and Cellier 2007)]. The mortality rate in patients with celiac disease exceeds that of the general population; however, there is a trend towards reduction in mortality after 1-5 years on a gluten-free diet [(Corrao, Corazza et al. 2001); Rubio-Tapia, et al. 2010)].

Following a completely gluten-free diet is, however, very challenging. Even highly motivated patients who try to maintain a strict dietary regimen are affected due to inadvertent or background exposure to gluten (FDA 2006). As many as 80% of patients with celiac disease who are in clinical remission and who claim to be following a gluten-free diet, have persistent abnormalities in small bowel biopsy specimens [(Lee and Newman 2003), (Bardella, Velio et al. 2007)]. Inadvertent exposure to gluten has been identified as the leading cause of non-responsive celiac disease among clinically diagnosed patients who were presumed to be on a gluten-free diet (Abdulkarim, Burgart et al. 2002). It is evident that a gluten-free diet is more expensive than a so-called ‘normal’ diet; also social life and travel contribute to dietary lapses. Taken together, there is an acute need for non-dietary therapies for celiac disease (Khosla, Gray et al. 2005).

As reviewed by Marsh and Crowe (Marsh and Crowe 1995), time-course studies of gluten challenges provide clear evidence of an inflammatory process, a dose-dependent accumulation of lymphocytes to the epithelium during the lower-dose challenges. Upon further challenge, crypt hyperplasia occurs and lastly, villus effacement is seen (flat mucosal lesion). As evidenced in clinical practice (silent celiac disease), also upon gluten challenge, the mucosal deterioration is often seen before clinical symptoms occur (Mäki, Landeaho et al. 1989). When challenging adolescents and young adults with 10 g of gluten per day, and performing a control small intestinal biopsy at the time of seroconversion of the celiac-type autoantibodies, it became evident that the gut mucosa relapsed in 70% of the patients before clinical symptoms occurred. Thus, gluten-induced damage in the small intestinal mucosa is a prerequisite for symptoms and complications of celiac disease, some of which may occur only years or decades after starting gluten ingestion.

Diagnosed celiac disease patients in Finland have typically had at least two upper gastrointestinal endoscopies with multiple biopsies, first at the initial diagnosis and the other approximately one year later to show gut mucosal healing upon a gluten-free diet (Management Model of Celiac Disease in Finland). The Celiac Disease Study Group in Tampere, Finland, has used small intestinal mucosal morphometric analyses to determine ingested wheat-, rye-, and barley-(gluten)-induced mucosal inflammation and mucosal architectural changes in early developing celiac disease, in active and clinically silent disease, during treatment follow-up of patients when the mucosa is healing [(Mäki 1991), (Holm, Mäki et al. 1992), (Holm 1993), (Iltanen, Holm et al. 1999) (Kaukinen, Collin et al. 1998), (Kaukinen, Collin et al. 1999), (Kaukinen 2000), (Kaukinen 2001), (Jarvinen, Kaukinen et al. 2003), (Peraaho, Kaukinen et al. 2003), (Jarvinen 2004), (Collin, Thorell et al. 2004), (Kaukinen 2005), (Salmi 2006)], as well as during gluten challenge (Holm, Maki et al. 2006). These parameters have included determination of the villus height/crypt depth ratio to establish manifest gluten-induced mucosal architectural change, and counting of the intraepithelial densities of all T lymphocytes (CD3-positive IELs), and densities of αβ+ and γδ+ T cell receptor-bearing IELs to reveal gluten-induced inflammatory changes.

People eating an average Western diet ingest approximately 15-25 g gluten per day. In celiac disease, the onset of symptoms and signs of gluten intolerance may occur in childhood but become evident most often only in adulthood or in the elderly after decades of gluten ingestion. It has been shown in previous clinical gluten challenge studies that older children, adolescents and young adults with long-term treated celiac disease can tolerate well the ingestion of 10-20 g gluten per day [(Mäki, Ländeaho et al. 1989), (Holm, Mäki et al. 2006)]. Also, a gluten challenge with repeated small intestinal mucosal biopsies has until fairly recently been mandatory to establish the definite diagnosis of celiac disease, especially in children (in some parts of the world this regimen is still followed). The effect of small gluten loads on the mucosal integrity and a safe gluten threshold in treated celiac disease is still under discussion [(Peraaho, Kaukinen et al. 2003), (Collin 2004) (Catassi, Fabiani et al. 2007), (Hischenhuber 2005)]. The literature indicates that doses of 1.5 to 2 g of gluten per day should cause some deterioration and inflammation but without inducing too many clinical symptoms and causing severe side effects. One and a half grams to 2 g of daily gluten exposure corresponds to the ingestion of approximately one-half to two-thirds a slice of wheat flour-based bread per day. A drug, to be clinically effective, should be able to significantly reduce or prevent the mucosal deterioration caused by a daily gluten challenge.

The only currently available treatment option for celiac disease patients is complete exclusion of dietary gluten; however, because gluten is found ubiquitously in the food supply, strict avoidance is extraordinarily difficult if not impossible for most patients. Because of ongoing gluten exposure, celiac disease patients (even when attempting to adhere to a gluten-free diet) suffer from the consequences of continued gluten exposure, including a significant increase in associated morbidity and mortality. Taken together, these observations establish that celiac disease represents a serious unmet medical need; therefore, a therapeutic intervention that could serve as an adjunct to an attempted gluten-free diet to attenuate or eliminate the pro-inflammatory, immunogenic potential of gluten in celiac disease patients would be a major clinical advance in the treatment of this disease. The ultimate goal in celiac disease clinical research is to prevent disease and sustain health, and also to develop new therapeutic strategies, less burdensome than a strict life-long gluten-free diet (Sollid and Khosla 2005). In this way it is hoped that celiac disease patients may in the future be able to ingest foods containing wheat, barley, and rye. In searching for alternative treatments for celiac disease, it is evident that patients have to undergo repeated upper gastrointestinal endoscopies and gluten challenges. A drug for the treatment of celiac disease should be able to prevent gluten-induced mucosal injury. Only then will celiac experts, advisors for celiac support groups, and patient organizations, accept the drug as an adjunct therapy or alternative treatment to strict gluten-free diet.

### Proteases for the Treatment of Celiac Disease

A promising new approach to treating celiac disease involves the oral administration of proteases, called glutenases, which can degrade gluten. See PCT Pat. Pub. No. 2003/068170; 2005/107786; 2007/044906; 2007/047303; 2008/115411; and 2010/021752; and U.S. Pat. Nos. 7,303,871; 7,320,788; 7,628,985; 7,910,541; and 7,943,312, each of which is expressly incorporated herein by reference.

Cysteine endoprotease (EP) B2 (also known as EPB2), a barley derived protease, and other similar proteases derived from the germinating seeds of the gluten-containing cereals have been identified as effective agents for the detoxification of gluten, the causative agent in celiac disease (including Celiac sprue and dermatitis herpetiformis; see U.S. Pat. No. 7,303,871, incorporated herein by reference). A modified, recombinant form of the barley-derived EPB2 zymogen called “ALV001” (the active form of this enzyme is termed “ALV001* herein) has been used as part of a combination enzyme therapy (including a prolyl endopeptidase (PEP), such as Sphingomonas capsulata PEP) for oral administration to celiac disease patients to aid in the digestion of gluten before it can exert its toxic effects in these patients (see U.S. Pat. No. 7,320,788; U.S. Pat. App. Pub. No. 20080193436; PCT Patent Pub. Nos. 2008/115428; 2008/115411; 2010/021752; and 2010/042203, each of which is expressly incorporated herein by reference). The ALV001 zymogen becomes active (converts to ALV001*) below pH 5, but is not activated at a higher pH.

ALV003 is an especially promising new drug in clinical development that is a mixture of two glutenases. See PCT Pat. Pub. Nos. 2005/107786; 2008/115428; 2008/115411; 2010/021752; and 2010/042203, each of which is expressly incorporated herein by reference. Oral glutenases such as ALV003 help to proteolyze the immunoreactive gluten peptides present in food before they can trigger an immune response in the intestinal mucosa [(Cerf-Bensussan, Matysiak-Budnik et al. 2007), (Piper, Gray et al. 2004), (Gass, Vora et al. 2006), (Pyle, Paaso et al. 2005), (Sollid and Khosla 2005), (Stepniak, Spaenij-Dekking et al. 2006)]. There remains a need for new methods and pharmaceutical compositions that can be used to protect celiac disease patients and other individuals suffering from gluten intolerance from the harmful effects of inadvertent exposure to gluten and to make gluten ingestion safer for them. The present invention meets these needs.

### SUMMARY OF THE INVENTION

In a first aspect, the present invention provides new pharmaceutical compositions of ALV001 and/or ALV001*, ALV002, and ALV003 (an orally administered, fixed dose (1:1 ratio by weight) combination of ALV001 and/or ALV001* and ALV002; see PCT Pub. No. 2008/115411, incorporated herein by reference), and new unit dose forms containing such compositions.

In one embodiment, the components of the formulated ALV001 drug substance are the ALV001 proenzyme and/or its active form ALV001*, and the excipients mannitol, TRIS, sucrose, EDTA, sodium chloride, citric acid, sodium metabisulfite, cysteine and monothioglycerol. In one embodiment, the components of the formulated ALV002 drug substance are the ALV002 enzyme and the excipients mannitol, TRIS, EDTA, sodium phosphate, calcium carbonate, and monothioglycerol.

In one embodiment, a patient in need of treatment for a condition as described herein is administered a daily dose of ALV003, which dose may be delivered by separate administration of ALV001 and/or ALV001* and ALV002, ranging from 100 mg to 3 g each. The daily dose may be subdivided into two, three, or more separate doses (“subdivided daily dose”). The daily dose or each subdivided daily dose is taken with a meal. Patients can take a dose or subdivided daily dose with any meal, with every meal, with meals known to the patient to contain gluten, and/or with meals unknown to the patient to contain gluten. Patients can maintain therapy for any period, such as a day, a week, a month, a year, a decade, and their entire life. Intermittent therapy can also be practiced by the patient.

Patients with conditions suitable for treatment in accordance with the invention include: celiac disease (Celiac sprue and/or dermatitis herpetiformis) patients, including patients with active disease and patients with disease in remission; patients requiring protection from a sign or symptom of celiac disease, including but not limited to a skin lesion or intestinal mucosal injury due to gluten ingestion up to 250 mg, 500 mg, 1 g, 2 g, 3 g, 5 g, 10 g, or 25 g or more gluten per day; patients who need therapy for an existing sign or symptom of celiac disease, including but not limited to a skin lesion or intestinal mucosal injury due to gluten ingestion up to 250 mg, 500 mg, 1 g, 2 g, 3 g, 5 g, 10 g, or 25 g or more gluten per day; undiagnosed celiac disease; patients with gluten-intolerance; and persons simply wishing to avoid gluten ingestion and accelerate digestion of any gluten ingested from their diet. In some embodiments, the patient needs therapy for an existing intestinal mucosal injury, and treatment results in healing as evidenced by improvement in the patient's Vh:Cd.

Without limitation, the invention includes the following particular aspects and embodiments:

In one aspect, the invention concerns a method for protecting a patient from a deleterious effect of gluten ingestion, said method comprising administering to the patient a dose of ALV003 sufficient to prevent said deleterious effect.

In another aspect, the invention concerns a method for preventing signs or symptoms of celiac disease in a celiac disease patient ingesting gluten, the method comprising orally administering ALV003 in an amount ranging from 150 mg to 3 g per day.

In another aspect, the invention concerns use of ALV003 in the preparation of a medicament for protecting a patient from a deleterious effect of gluten ingestion, by administering to said patient a dose of ALV003 sufficient to prevent said deleterious effect.

In yet another aspect, the invention concerns use of ALV003 in the preparation of a medicament for preventing signs or symptoms of celiac disease in a celiac disease patient ingesting gluten, by orally administering ALV003 in an amount ranging from 150 mg to 3 g per day.

In a further aspect, the invention concerns ALV003 for use in protecting a patient from a deleterious effect of gluten ingestion, by administering to said patient a dose of ALV003 sufficient to prevent said deleterious effect.

In a still further aspect, the invention concerns ALV003 for use in preventing signs or symptoms of celiac disease in a celiac disease patient ingesting gluten, by orally administering ALV003 in an amount ranging from 150 mg to 3 g per day.

In a different aspect, the invention concerns a kit comprising ALV003 in a container and a label affixed to or instructions associated with the container directing administration of said ALV003 to protect a patient from a deleterious effect of gluten ingestion.

In a further aspect, the invention concerns a kit comprising ALV003 in a container and a label affixed to or instructions associated with the container directing administration of said ALV003 to prevent signs or symptoms of celiac disease in a celiac disease patient ingesting gluten, by orally administering ALV003 in an amount ranging from 150 mg to 3 g per day. The invention encompasses a method for protecting a patient from a deleterious effect of gluten ingestion, the method comprising administering to the patient a dose of ALV003 sufficient to prevent said deleterious effect. In this and any of the foregoing embodiments, the ALV003 may be in a form in which the ALV001 (or ALV001*) is in a separate dosage form from the ALV002 or in a form in which the ALV001 (or ALV001*) and ALV002 are admixed or otherwise combined in a single unit dosage form.

The embodiments herein are equally applicable to all aspects of the invention, including the particular aspects enumerated above.

In one embodiment, the patient has celiac disease, and said deleterious effect is intestinal mucosal injury.

In another embodiment, the patient has symptomatic celiac disease.

In yet another embodiment, the patient is moderately to severely symptomatic.

In a further embodiment, the patient has experienced symptoms of celiac disease, ranging from moderate to severe, within one month prior to said first administration.

In certain embodiments, the symptoms are self reported, where the patient may report, for example, symptoms with a severity score of at least 3 on a 0-10 numeric rating scale of severity, or with a severity score of at least 4 on a 0-10 numeric rating scale of severity.

In other embodiments, the serology status of the patient is determined prior to administration, where determination of the serology status may comprise, without limitation, an antibody test selected from the group consisting of anti-gliadin antibodies (AGA), anti-reticulin antibodies (ARA), IgA anti-human tissue transglutaminase (TTG) antibodies (TG2), IgA anti-endomysial antibodies (EMA), and anti-deamidated gliadin peptide (DPG) tests.

In a further embodiment, the patient does not exhibit IgE-mediated reaction to wheat.

In various embodiments, administration may occur at mealtime, such as with a major meal, for example, with major meals or at least one to three times per day. In various embodiments, administration may occur at any time food suspected of containing gluten is ingested by the patient.

In various embodiments, the signs or symptoms of celiac disease comprise one or more of diarrhea, constipation, abdominal pain, bloating, nausea, fatigue, and skin rash.

In other embodiments, the ALV003 dose administered may, for example, be in the range of 150 mg to 1.5 g per administration, i.e., 900 mg per administration.

In further embodiments, the dose may be administered at least once a day for at least a month, or at least 300 days per year, for at least two years.

In one embodiment, each dose of ALV003 comprises a dose of ALV001 and/or ALV001* in powdered form and a dose of ALV002 in powdered form, and said powders are dissolved in a potable liquid to be ingested by said patient.

In various embodiments, the ALV003 dose is administered with food containing at least 20 mg but not more than 25 g of gluten, or with food containing no more than about 1 g of gluten, or with food containing no more than about 2 g of gluten, or with food containing no more than about 3 g of gluten, or with food containing no more than about 5 g of gluten, or with food containing no more than about 10 g of gluten, or with food containing no more than about 15 g of gluten, or with food containing no more than about 25 g of gluten.

In a further embodiment, the ALV003 has equal amounts of ALV001 and/or ALV001* and ALV002, wherein ALV001 and/or ALV001* has a specific activity of at least 5000 or more proteolytic activity units per mg, and said ALV002 has a specific activity of at least 3000 or more proteolytic activity units per mg.

In a further aspect, the invention concerns a kit comprising ALV003 in a container and a label affixed to or instructions associated with the container directing administration of said ALV003 to protect a patient from a deleterious effect of gluten ingestion.

In a still further aspect, the invention concerns a kit comprising ALV003 in a container and a label affixed to or instructions associated with the container directing administration of said ALV003 to prevent signs or symptoms of celiac disease in a celiac disease patient ingesting gluten, by orally administering ALV003 in an amount ranging from 150 mg to 3 g per day.

In another aspect, the invention concerns a unit dosage form of ALV003 for oral administration. In various embodiments, the unit dosage form may, for example, be a table, a powder, sachet(s) comprising powdered forms of one or more ingredients, or a liquid.

Thus, the unit dosage form may comprise one or more sachets comprising a powdered form of ALV001 and/or ALV001* and/or ALV002.

In one embodiment, the unit dosage form comprises ALV001 and/or ALV001* and ALV002 in the same sachet.

In another embodiment, the unit dosage form comprises ALV001 and/or ALV001* and ALV002 in separate sachets.

In other embodiments, the unit dosage form may further comprise a flavor agent, which may, for example, be contained in a sachet comprising ALV001/and/or ALV001* and/or ALV002, or may be formulated separately, e.g. in a separate sachet.

Optionally, the unit dosage forms may contain instructions for dilution of the powder or powders contain in the sachet(s), which may be dissolved in a potable liquid and reconstituted as a drink. The potable liquid may, for example, be water or fruit juice.

All embodiments can be variously combined with each other, and, as noted before, apply to all aspects of the invention.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 illustrates the patient disposition in a Phase 2a double-blind, placebo-controlled trial in celiac disease patients to demonstrate the ability of ALV003 to inhibit gluten-induced ill health using a study design of 6-weeks challenge with gluten.

FIG. 2 shows the demographics of the patients participating in the Phase 2a clinical trial.

FIG. 3 shows the baseline characteristics of the patients participating in the Phase 2a clinical trial.

FIG. 4 shows the Villus Height (Vh):Crypt Depth (Cd) ratios in the treatment and placebo patient population.

FIG. 5 shows the proportion of patients with little or no change in Vh:Cd.

FIG. 6 shows the quantification of CD3+ T cells (cells/mm) in the treatment and placebo patient population at the beginning of the treatment (baseline) and at week 6.

FIG. 7 shows the quantification of αβ T cells (cells/mm) in the treatment and placebo patient population at the beginning of the treatment (baseline) and at week 6.

FIG. 8 shows the quantification of γδ T cells (cells/mm) in the treatment and placebo patient population at the beginning of the treatment (baseline) and at week 6.

FIG. 9 presents a summary of additional observations resulting from the Phase 2a clinical trial, including discussion of serologic endpoints, adverse events, and Gastrointestinal Symptom Rating Scale (GSRS).

FIG. 10 shows the 401 amino acid sequence of the recombinant form of ALV001 (SEQ ID NO: 1), which is a modified version of a proenzyme form of cysteine endoprotease B, isoform 2 (EP-B2), naturally occurring in germinating barley, Hordeum vulgare. The naturally occurring signal sequence of the barley gene product was deleted. Residues in bold are derived from the recombinant expression vector. Underlined residues were introduced during cloning. The downward arrow, “↓”, represents the N-terminus of mature EP-B2 following enzyme activation (Bethune et al., 2006). The bold sequences on the N-terminus and C-terminus contain hexa-His tags, which have high affinity for nickel affinity resins and were introduced to facilitate purification prior to protein refolding.

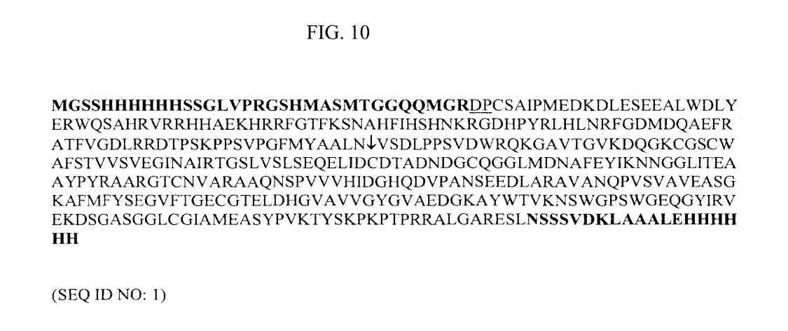

FIG. 11 shows the amino acid sequence of ALV001* (SEQ ID NO: 2), which is the mature form of ALV001 (SEQ ID NO: 1) shown in FIG. 10, having a truncated N-terminus that begins after the downward arrow.

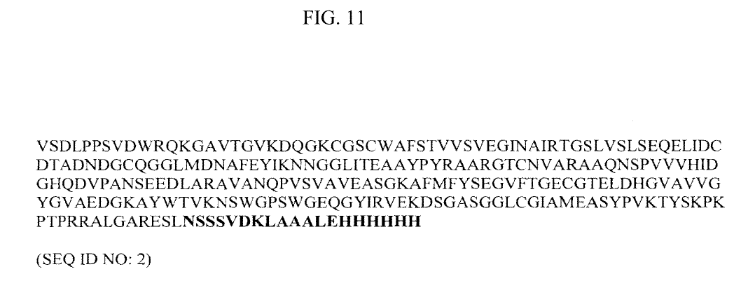

FIG. 12 shows the 741 amino acid sequence of the recombinant form of ALV002 (SEQ ID NO:3). The gene that naturally encodes SC-PEP was engineered with the following two significant features to generate the ALV002 gene: (1) codons of the gene have been optimized for translation in E. coli; and (2) nucleotides derived from the expression vector were added to the N-terminus. An expression vector derived hexa-His tag near the N-terminus is removed along with 38 N-terminal amino acids through proteolytic processing of the enzyme during expression and purification. The downward arrow, “↓”, represents the location of proteolytic processing, and N-terminal amino acids derived from the recombinant expression vector, including the hexa-His tag, are shown in bold.

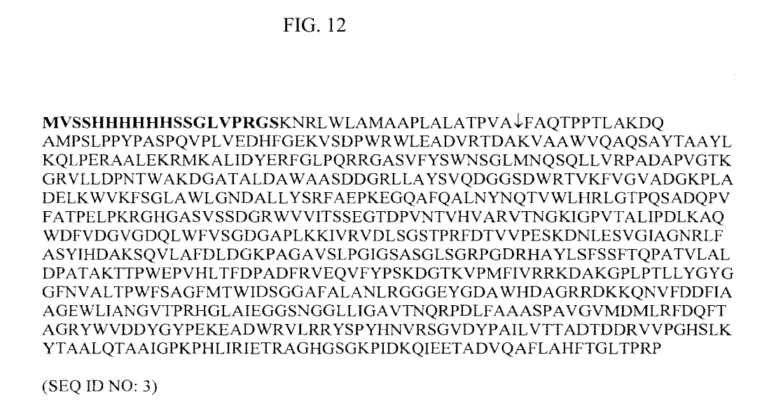

### DETAILED DESCRIPTION OF THE INVENTION

### I. Definitions

The term “ALV-1” or “ALV001” is used herein to refer to a zymogenic proenzyme form of cysteine endoprotease B, isoform 1 (EP-B2), naturally occurring in barley. The term, as used herein, specifically includes the polypeptide of SEQ ID NO: 1, with or without the highlighted, vector-derived N- and/or C-terminal residues and with and without the His tags incorporated in the N- and/or C-terminal sequences. The definition further includes post-translational modifications of the proenzyme. In the Examples, “ALV001” is used to refer to the recombinant form of the proenzyme.

The term “ALV-1*” or “ALV001*” is used herein to refer to an active form of the proenzyme ALV-1, as hereinabove defined. The term, as used herein, specifically includes the polypeptide of SEQ ID NO: 2, with or without the highlighted vector-derived C-terminal residues and with or without the C-terminal His tags. In the Examples, “ALV001*” is used to refer to the recombinant form of the active enzyme.

The term “ALV-2” or “ALV002” is used herein to refer to a recombinant version of a prolyl endopeptidase from the bacterium Sphingomonas capsulata (SC-PEP). The term, as used herein, expressly includes the 741 amino acid commercial form of ALV002 (SEQ ID NO:3), with or without the six contiguous histidine residues (hexa-His tag) added in the N-terminal region, and with or without the 38 N-terminal amino acids removed during proteolytic processing. In the Examples, “ALV001” is used to refer to the recombinant form of the enzyme.

The term “ALV-3” or “ALV003” is used herein to refer to a combination and/or co-administration of ALV-1 and ALV-2 or ALV-1* and ALV-2 or (ALV-1 and ALV-1*) and ALV-2 in a 1:1 (w/w) ratio. In one embodiment, ALV-1 and ALV-2 or ALV-1* and ALV-2 or (ALV-1 and ALV-1*) and ALV-2 are present in the same formulation/dosage form in 1:1 (w/w) ratio (and the formulation/dosage form may include either a formulation in which the two enzymes are admixed or otherwise combined in a single unit dosage form or a formulation in which the two enzymes are in separate dosage form for co-administration). Unless expressly indicated otherwise, the term “ALV-3” or “ALV003” includes combinations comprising ALV-1 and/or ALV-1*. In the Examples, “ALV003” is used to refer to a combination or co-administration of the recombinant forms of ALV001 and/or ALV001* and ALV002.

The term “ALV001/ALV001*” is used herein to mean that either ALV001 or ALV001* can be used.

The terms “simultaneous administration,” “co-administration” and “concurrent administration” are used interchangeably and refer to the administration of at least two active agents, using separate formulations or a single pharmaceutical formulation, and consecutive administration in either order, wherein preferably there is a time period while both (or all) active agents simultaneously exert their biological activities. Thus, the second therapeutic agent, such as ALV-2 may be administered prior to, contemporaneously with, or following, administration of the first therapeutic agent, such as ALV-1 and/or ALV-1*. In one particular embodiment, the two or more active agents (such as ALV-1 and/or ALV-1* and ALV-2) are administered to the patient the same time, in a single formulation, i.e., a single unit dosage forms, or separate formulations of the two enzymes, i.e., two different dosage forms that are administered concurrently to the patient.

The terms “celiac sprue” and “celiac disease” are used interchangeably and refer to an autoimmune disease of the small intestine caused by the ingestion of gluten proteins from widely prevalent food sources such as wheat. The terms, as used herein, specifically include clinically silent celiac disease, characterized by absence of gastrointestinal symptoms, and moderate to severe symptomatic celiac disease, characterized by gastrointestinal symptoms that can range from mild to severe. “Celiac disease” as used herein also includes dermatitis herpetiformis.

The term “deleterious effect of gluten ingestion” is used herein to refer any and all undesired effects of gluten injestion in a subject, including, without limitation, symptoms and deleterious effects resulting from T lymphocyte-driven immune response in the intestine of celiac disease patients, including gastrointestinal symptoms, such as gluten-induced small intestinal mucosal inflammation and symptoms. The term “deleterious effect of gluten ingestion” also includes any undesired effects of gluten ingestion on the skin of a subject, including, without limitation, symptoms characteristic of dermatitis herpetiformis.

### II. Detailed Description

Activity Assays

For ALV001* activity measurement, ALV001* samples are diluted into a volume of 50 μL of dilution buffer (100 mM Tris, 3.5 mM EDTA, 2 mM β-mercaptoethanol, 15% w/v sucrose, 5 mg/mL bovine serum albumin, pH 8.0) to a concentration of 400 nM. This volume is added to 100 μL of 1M sodium acetate buffer (pH 4.5), and incubated at 30° C. to allow for enzyme activation. After exactly 30 min., the entire volume is added to a cuvette containing 850 μL substrate (Z-Phe-Arg-pNA) solution in 5% (v/v) DMSO/H₂O (50 μM substrate concentration in final assay volume). The reaction is immediately followed by monitoring A410 at 25° C. with a UV/Vis spectrophotometer. The reaction rate is measured from the initial slope of the A410 versus time, and converted to activity units using extinction coefficient of 8,800 M⁻¹ cm⁻¹ for pNA. One unit is defined as the amount of ALV001* required to release 1 μM pNA per minute in above reaction conditions.

Similarly for ALV002 activity measurement, ALV002 samples are diluted into a volume of 100 μL of dilution buffer (100 mM Tris, 3.5 mM EDTA, 2 mM 1-thioglycerol, 2.5% w/v mannitol, 5 mg/mL bovine serum albumin, pH 8.5) to a concentration of 100 nM. This entire volume is added to a cuvette containing 900 μL substrate (Z-Gly-Pro-pNA) solution in 2.8% (v/v) DMSO/H2O and 22.2 mM sodium phosphate, pH 7.0 (50 μM substrate concentration in final assay volume). The reaction is immediately followed by monitoring A410 at 25° C. with a UV/Vis spectrophotometer. The reaction rate is measured from the initial slope of the A410 versus time, and converted to activity units using extinction coefficient of 8,800 M⁻¹ cm⁻¹ for pNA. One unit is defined as the amount of ALV002 required to release 1 μM pNA per minute in above reaction conditions.

ALV003 Mechanism of Action

Gluten has a high proline and glutamine content. This makes it resistant to proteolysis by gastric, pancreatic, and intestinal brush border endo- and exoproteases, which have poor specificity for peptide bonds adjacent to proline and glutamine residues (See (Hausch, Shan et al. 2002), (Shan, Molberg et al. 2002), (Piper, Gray et al. 2004)). As a consequence of the incomplete gastrointestinal proteolysis of gluten, long oligopeptides (such as the 33-mer and 26-mer peptide fragments) accumulate in the small intestine of mammals following ingestion of gluten. Following deamidation by tissue transglutaminase in the intestine, these peptides stimulate an immune response in the intestine of celiac disease patients resulting in structural changes to the lining of the small intestine (Kagnoff 2007). Following the seminal work by Khosla et al. described in PCT Pat. Pub. No. 2003/068170, a number of scientific journal publications have reported the potential for proline- and glutamine-specific endoproteases, referred to as glutenases, as therapeutic agents for celiac disease because of their ability to digest these proteolytically resistant gluten epitopes [(Marti, Molberg et al. 2005), (Shan, Qiao et al. 2005), (Bethune, Strop et al. 2006), (Gass, Vora et al. 2006), (Siegel, Bethune et al. 2006), (Cerf-Bensussan, Matysiak-Budnik et al. 2007), (Gass, Bethune et al. 2007)].

ALV003 is a mixture of two glutenases. The two glutenases that are comprised in ALV003 demonstrate complementary substrate sequence and chain length specificity. If ALV003 comprises the proenzyme form of EPB2, ALV001, upon activation in an acidic environment (as in the stomach) to form ALV001*, proteolyzes gluten at specific glutamine residues and reduces the amount of peptides that are immunostimulatory to T cells derived from celiac disease patients [(Siegel, Bethune et al. 2006), (Bethune, Strop et al. 2006)]. Although ALV002 alone has relatively weak activity on intact gluten proteins, it proteolyzes the peptidic products of ALV001 digestion by cleaving at proline residues [(Shan, Marti et al. 2004), (Gass, Bethune et al. 2007)]. By virtue of their complementary sequence specificity and chain length tolerance for peptides, together ALV001/ALV001* and ALV002 degrade gluten more rapidly and thoroughly than either individual enzyme alone (Gass, Bethune et al. 2007).

The complementary substrate sequence and chain length specificity described above have been demonstrated in vitro and in vivo. ALV003 proteolyzes various forms of gluten (purified gliadin, uncooked gluten flour, and whole wheat bread gluten) in vitro and eliminates >90% of immunoreactive epitopes present. In addition, ALV003 proteolyzed both gluten flour and wheat bread in the stomach of a rat in an in vivo model of gluten digestion. As noted above, at pH values typical of a postprandial stomach (3.5-5), ALV001 activates to its mature form ALV001*, which is active and stable over this pH range. ALV002 contributes to gluten digestion above pH 4. Therefore, ALV003 is active in the stomach during and following a meal. In addition, ALV003 is rapidly proteolyzed by pepsin in both simulated and fasting human gastric fluid (pH 1.8) and also by pancreatin at near neutral pH, providing a mechanism for ALV003 clearance. When incubated in human gastric samples obtained from subjects who had ingested soy milk ex vivo, ALV003 degraded gluten immunoreactive epitopes measured within 30 minutes in a dose-dependent fashion. In vitro, concentrations of ALV003 from 0.25-2.0 mg/mL were able to eliminate >90% of immunoreactive gluten peptides from 0.5-12 mg/mL gluten within 60 min.

Notable favorable properties of ALV003 include its high specificity for gluten and suitability for convenient oral dosing.

Pharmaceutical Compositions

Although the following description refers to “ALV001”, in some embodiments of the pharmaceutical formulations and unit dosage forms of the invention the activated form of ALV001, ALV001*, is used instead of, or in addition to, the inactive zymogen. The following description equally applies to pharmaceutical compositions comprising ALV001 and/or ALV001*.

In various embodiments of the pharmaceutical compositions of the invention, a reducing agent is included to enhance stability. For example, the reducing agents sodium metabisulfite (MBS) and cysteine have been shown to maintain ALV001 activity in food using two independent assays, mass spectrometry and ELISA, which were used to quantify the ability of ALV001 to degrade gluten in the presence of a complex meal. Results showed that cysteine (100 mg per meal), in the absence of MBS, maintained linearity of enzyme activity over time, resulting in a 20-fold increase in the ability of ALV001 to degrade gluten over 30 minutes. Cysteine increased ALV001 stability an additional 3-fold in a complex meal containing MBS (8 mg per meal) and, unlike MBS alone, stabilized ALV001 activity in the presence of specific foods. One hundred milligrams of cysteine per meal were required to achieve maximal ALV001 stability under the test conditions employed. Cysteine levels less than 100 mg provided less than maximal ALV001 stability under the test conditions employed. Mechanistically, MBS and cysteine are included in certain pharmaceutical compositions of the invention to counter the oxidative effects on ALV001 that derived from the food. Pharmaceutical formulations of ALV001 and ALV003 of the invention may therefore contain cysteine (e.g., about 100 mg, although more or less cysteine may be employed, depending on the amount of ALV001 in the dosage form) or MBS (e.g., about 8 mg although more or less MBS may be employed, depending on the amount of enzyme in the dosage form), or both cysteine and MBS.

A dosage form of the invention may, for example, contain 100 mg to 1 g ALV003 (e.g., 50 mg of each of ALV001 and ALV002 to 500 mg or each of ALV001 and ALV002), in powder or optionally in a tablet form, optionally with added mannitol, microcrystalline cellulose, sodium metabisulfite, and/or cysteine. The dosage form may contain a lyophilized powder of ALV001 in which sodium metabisulfite had been added to the solution prior to lyophilization at a ratio from 100:8 (w/w ALV001:MBS) to a ratio of 900:4 (w/w).

A dosage form may also contain a spray dried powder of ALV001 in which sodium metabisulfite had been added to the solution prior to spray drying at a ratio from 100:8 (w/w) to a ratio of 900:4 (w/w).

In another embodiment, a dosage form may contain ALV001 in a range of 50 to 1000 mg, e.g. 100 to 900 mg, and sodium metabisulfite in a range of 1 to 100 mg, e.g., 5 to 25 mg, or 1 to 10 mg.

Another dosage form may contain pulsed release ALV001 (or ALV002 or both) in a range of 50 to 1000 mg, where about half of the dose is immediately released upon ingestion and the remainder is released in a second pulse 20 minutes to 1 hour after ingestion.

In other embodiments, a dosage form may contain a protease (ALV001 or ALV002 or both) and a quantity of an antioxidant that achieves an antioxidant concentration of at least 30, 50, 100, or 200 μM in the stomach.

In further embodiments, the dosage form contains ALV001 and/or ALV002 and a quantity of a compound that generates a concentration of free thiol of at least 100, 200, or 500 μM in the stomach.

In further embodiments, the dosage form contains both ALV001 and ALV002 where one or both enzymes are formulated to provide an immediate release and the other enzyme or remainder of both enzymes is released either in a pulsed release or a controlled release.

All of the ALV001 dosage forms above can also be modified to include a second protease, such as a prolyl endopeptidase (PEP). In one embodiment, the PEP is Sphingomonas capsulata PEP, for example and without limitation, as described in PCT Pub. No. 2008/115411. In various embodiments, between 1-2000 mg of the PEP and ALV001 are dosed at a PEP:ALV001 weight ratio of between 1:100 to 100:1, more preferably between 1:20 to 20:1, more preferably between 1:5 and 5:1, and most preferably at a 1:1 w/w ratio. In one embodiment, the dosage form is constructed so that the ALV001 and antioxidant, e.g., sodium metabisulfite, are immediately released, and the PEP is released either immediately; or is released in one or more short, delayed pulses (from 10 minutes to 2 hours); or is released in sustained release over 10 minutes to 2 hours. In the ALV003 dosage forms, the antioxidant may be an antioxidant other than or in addition to sodium metabisulfite. Thus, the additional or other antioxidant may, for example, be selected from the group consisting of sodium sulfite, sodium bisulfate, potassium bisulfate, potassium metabisulfite, alone or combination; sodium thiosulfate; glutathione, cysteine, homocysteine, sodium dithionite, thioglycerol; and acetylcysteine.

The compositions herein may, for example, be in the form of roller compacted granules or pellets of the enzyme. The antioxidant may be either contained in the granules or pellets or blended with the granules or pellets and either filled or compressed into a dosage form.

In general, the pharmaceutical formulations used in accordance with the present invention can be in the form of, for example and without limitation, particles, particles in capsule or sachet, or tablet. Tablets may be single layer, bilayer, or multilayer and may be coated or uncoated. The formulation can, for example and without limitation, be added to a food or drink and then administered, for example, as a sprinkled powder or granule formulation or as a spread in the form of a jam or powder. A capsule of low water content may be desired for stability, and hypromellose capsules, HPMC, of size 1, 0, or 00, can be used. Capsules can be packaged in a dry environment either with desiccant or desiccant packs or if in blisters under dry nitrogen or other dry environment.

The pharmaceutical formulations used in accordance with the present invention can comprise a lubricant such as magnesium stearate, stearic acid, sodium stearyl fumarate, or sodium stearyl lactylate, hydrogenated vegetable oil (such as hydrogenated and refined triglycerides of stearic and palmitic acids). These may be at 0.3 to 5% of weight of the dosage form. If mannitol is contained at a high concentration in the lyophilized powder, then higher concentrations of lubricant may be used.

The protease powder may be blended with lubricant or other excipients such as a filler or binder and granulated. If the protease is unstable with water and temperature, then these can be roller compacted into granules, if necessary using chilled rollers for stability. One may optionally include an agent that modifies or controls pH, at least for the first few minutes after the dosage form is in the GI tract, to facilitate activation of zymogen proteins such as ALV001.

Fillers such as dicalcium phosphate, microcrystalline cellulose, maltodextrins, mannitol, lactose, sucrose, or trehalose may be included and blended with the powders or included in the lyophilized powder or spray-dried powder to prepare a pharmaceutical formulation of the invention. More hydrophilic fillers such as microcrystalline cellulose may be avoided for certain enzymes, such as ALV001.

Controlled-release excipients may be blended in to form polymeric drug-containing matrices. These matrices may be from about 1 mm in diameter to the size of a full tablet 10 to 12 mm in width and even 1.8 cm or more in length. These matrices can provide extended-release into the stomach being retained with food for 20 minutes to several hours depending on the size. These matrices may or may not be swellable. If swellable, extended-release hydrophilic polymers that are appropriate include cellulose polymers and their derivatives (such as for example, hydroxyethylcellulose, hydroxypropylcellulose, carboxymethylcellulose, and microcrystalline cellulose, polysaccharides and their derivatives, polyalkylene oxides, polyethylene glycols, chitosan, polyvinyl alcohol), xanthan gum, maleic anhydride copolymers, polyvinyl pyrrolidone), starch and starch-based polymers, poly (2-ethyl-2-oxazoline), poly(ethyleneimine), polyurethane hydrogels, and crosslinked polyacrylic acids and their derivatives. Further examples are copolymers of the polymers listed in the preceding sentence, including block copolymers and grafted polymers. Extended-release coatings could also be prepared on these particles using some of the above polymers.

In one embodiment, the pharmaceutical composition is a powder. In this embodiment, the powdered forms of ALV001 and ALV002 may be separately packaged, in sachets for example, and contemporaneously administered to the patient, or the powdered forms of the two proteases may be admixed prior to administration.

In one embodiment, the pharmaceutical composition is a powder formulation of ALV001 that can be prepared in a unit dose form containing from about 50 mg to about 1000 mg, e.g. 450 mg, of ALV001 (typically ≥5000 proteolytic activity Units/mg protein, i.e., 7300 to 7500 Units/mg, but the specific activity can be lower, i.e., about 2500 Units or even lower, depending on a variety of factors, such as the patient and the intended application) with any of the excipients described herein, including but not limited to those shown in the table below.

In one embodiment, the pharmaceutical composition is a powder formulation of ALV002 that can be prepared in a unit dose form containing 50 mg to about 1000 mg, e.g. 450 mg, of ALV002 (typically ≥3000 proteolytic activity Units/mg protein, i.e., 6200-7000 Units/mg protein, but the specific activity can be lower, i.e., about 1500 Units or even lower, depending on a variety of factors, such as the patient and the intended application) with any of the excipients described herein, including but not limited to those shown in the table below.

The protein (ALV001/ALV001* and ALV002) content of the compositions may vary within a wide range, such as, for example, between about 10% and about 80%, or between about 20% and about 70%, wherein ALV001/ALV001* and ALV002 may be present in equal or different percentage amounts. In one embodiment, the compositions contain about 20% ALV001 and about 30% ALV002. In another embodiment, the compositions contain about 70% of each of ALV001* and ALV002.

Specific compositions for ALV001 and ALV002 formulated drug substances are provided below:

ALV001 Formulated Drug Substance

| Component | Quantity per dose/bottle |
|---|---|
| ALV001 (EP-B2) | 450.0 mg ± 30.0 mg |
| EDTA | 69.9 mg ± 14.1 mg |
| Mannitol | 879.0 mg ± 175.8 mg |
| Monothioglycerol | 8.4 mg ± 1.8 mg |
| NaCl | 10.8 mg ± 8.1 mg |
| Sucrose | 351.6 mg ± 70.2 mg |
| TRIS | 508.2 mg ± 101.7 mg |
| Citric Acid Anhydrous | 450.0 mg ± 25.0 mg |
| Sodium Metabisulfite | 8.0 mg ± 1.0 mg |
| Cysteine Hydrochloride | 100.0 mg ± 10.0 mg |
| Monohydrate | |

ALV002 Formulated Drug Substance

| Component | Quantity per dose/bottle |
|---|---|
| ALV002 (PEP) | 450.0 mg ± 30.0 mg |
| EDTA | 32.7 mg ± 6.6 mg |
| Mannitol | 439.5 mg ± 87.9 mg |
| Monothioglycerol | 3.9 mg ± 0.9 mg |
| Sodium Phosphate | 10.8 mg ± 2.1 mg |
| TRIS | 238.2 mg ± 47.7 mg |
| Calcium Carbonate | 400.0 mg ± 25.0 mg |

Suitable ALV001 and ALV002 placebos contain the same ingredients as the corresponding formulated drug substances, except for the removal of monothioglycerol and EDTA and the addition of TRIS-HCl (trishydroxymethylaminomethane-HCl). All are free-flowing white to off-white powders. ALV001, ALV002, and matching placebos can be provided in separate polypropylene bottles and stored at room temperature (15 to 25° C.). It is also possible to fill the ALV001 and ALV002 powders into other types of containers, such as stickpacks.

ALV001 and ALV002 can also be combined in a single liquid preparation (drink).

All formulations may additionally contain one or more flavor agents, providing a variety of flavor choices.

In further specific embodiments, the ALV001* and ALV002 drug substances have the following compositions:

| | 100 mg | | 300 mg | | 450 mg | | 900 mg | |
|---|---|---|---|---|---|---|---|---|
| Active/Excipient | ALV001* | ALV002 | ALV001* | ALV002 | ALV001* | ALV002 | ALV001* | ALV002 |
| ALV001 | 50 | 0 | 150 | 0 | 225 | 0 | 450 | 0 |
| ALV002 | 0 | 50 | 0 | 150 | 0 | 225 | 0 | 450 |
| Tris | 1.3 | 1.9 | 3.8 | 5.8 | 5.8 | 8.6 | 11.5 | 17.3 |
| EDTA | 2.1 | 3.2 | 6.4 | 9.6 | 9.6 | 14.4 | 19.2 | 28.8 |
| Mannitol | 10.7 | 16 | 32 | 48 | 48 | 72 | 96 | 143.9 |
| MTG | 0.2 | 0.3 | 0.6 | 1 | 1 | 1.4 | 1.9 | 2.9 |
| Sodium | 0.7 | 0 | 2.2 | 0 | 3.4 | 0 | 6.7 | 0 |
| Citrate | | | | | | | | |
| Citric Acid | 6.4 | 0 | 19.2 | 0 | 28.8 | 0 | 57.6 | 0 |
| Cysteine HCl | 100 | 0 | 100 | 0 | 100 | 0 | 100 | 0 |
| Sodium | 8 | 0 | 8 | 0 | 8 | 0 | 8 | 0 |
| Metabisulfite | | | | | | | | |
| Buffer* | 1000 | 1000 | 1000 | 1000 | 1000 | 1000 | 1000 | 1000 |
| Sodium Stearyl | 24 | 21 | 26 | 24 | 29 | 26 | 35 | 33 |
| Fumarate | | | | | | | | |
| Flavor^{‡} | 24 | 21 | 26 | 24 | 29 | 26 | 35 | 33 |
| Total | 1227 | 1113 | 1374 | 1263 | 1488 | 1373 | 1821 | 1709 |
| Mass/Stickpack | | | | | | | | |

Optionally, the formulations may include one or more fillers, such as mannitol.

Optionally the formulations may contain a flavor agent, e.g. as shown in the table above, which may be present in the ALV001* and/or ALV002 formulation or may be separately packaged. When the formulation is prepared in the form of stickpacks, in one particular embodiment, the ALV001* and ALV002 stickpacks do not contain any flavor agent, rather the favor agent is present in a third stickpack, which may optionally contain one or more fillers, such as mannitol. Alternatively, for example, a portion of the buffer/dose can be used in the flavor-containing stickpack to add bulk.

Optionally, the amount of cysteine per dose can be reduced to improve taste.

Reconstitution of the powdered forms of the drug substances may take place using a potable liquid, such as, for example, cold or room temperature water or a fruit juice. In a specific embodiment, the formulations listed in the table immediately above will be reconstituted in cold or room temperature water.

Methods of Treatment

ALV003 and pharmaceutical compositions comprising ALV003 can be used in methods for protecting a patient from a deleterious effect of gluten ingestion. In particular, ALV003 can be used to prevent or treat celiac disease, including celiac sprue and/or dermatitis hepatiformis, including prevention and treatment of various symptoms or clinical manifestations of these diseases and conditions.

Clinical manifestations include, without limitation, mild gastrointestinal disturbances, chronic gastrointestinal symptoms, malabsorption, weight loss, isolated iron deficiency anemia, various manifestations outside the gut, such as osteoporosis, peripheral and central nervous system involvement, mild or severe liver disease, infertility problems, and the-gluten-induced skin disease, dermatitis herpetiformis. The gluten-induced small bowel pathology in celiac disease is characterized by an inflammatory reaction that is accompanied by villus atrophy and hypertrophy of crypts.

Symptoms of celiac disease include, without limitation, diarrhea, constipation, flatulence, abdominal pain, bloating, nausea, fatigue, skin rashes, difficulty thinking, and headache.

The patients may be symptomatic or asymptomatic at the time of treatment. If the patient is symptomatic, symptoms may range from mild through moderate to severe. In a particular embodiment, moderately to severely symptomatic celiac sprue patients are treated. In another embodiment, the treatment is used in moderately to severely symptomatic celiac disease patients as an adjunct to a GFD for the attenuation of gluten-induced small intestinal mucosal injury and symptoms. In another embodiment, the patient treated has experienced moderately to severe symptoms of celiac disease within one month from first administration.

The severity of the disease can also be determined using medical diagnostic methods known in the art, such as upper gastrointestinal endoscopies, biopsies, small intestinal mucosal morphometric analyses, determination of the villus height/crypt depth ratio to establish manifest gluten-induced mucosal architextural change, and measuring the intraepithelial densities of all CD3+ (T) lymphocytes and densities of αβ+ and γδ+ T cell receptor-bearing IELs to reveal gluten-induced inflammatory changes.

While thrice daily (TID) administration is contemplated in various embodiments of the invention, QD administration may also be practiced, i.e., when a patient is consuming only one gluten-containing (or potentially gluten-containing) meal per day. Thus, ALV003 may be administered when a patient is ingesting food suspected of containing, or known to contain, gluten.

Optionally, the patient's serology status may be determined prior to administration of the compositions herein. Determination of the serology status may comprise an antibody test, such as anti-gliadin antibodies (AGA), anti-reticulin antibodies (ARA), IgA anti-human tissue transglutaminase (TTG) antibodies (TG2), and IgA anti-endomysial antibodies (EMA), and anti-deamidated gliadin peptide (DGP) tests.

Administration may occur at mealtime, such as with a major meal or meals, e.g. one to three times, such as three times, per day.

A typical daily dose for oral administration of ALV003 is in the range of about 150 mg to about 3 g. As discussed earlier, the daily dose can be reached by one or more administrations, typically taken with food.

In various embodiments, ALV003 is administered with food containing at least 20 mg but not more than about 25 g of gluten, or no more than about 1 g of gluten, or no more than about 2 g of gluten, or no more than about 3 g of gluten, or no more than about 5 g of gluten, or no more than about 10 g of gluten.

In another embodiment, ALV003 has equal amounts of (ALV001 and/or ALV001*), and ALV002, by weight or by units of activity, including embodiments wherein ALV001/ALV-001* has a specific activity of at least 5000 or more proteolytic activity units per mg, and said ALV002 has a specific activity of at least 3000 or more proteolytic activity units per mg.

Further details of the invention will be illustrated by the following non-limiting examples.

### Example 1

ALV-003-0801

Study ALV003-0801 was a phase 0 study designed to compare the effects of gluten treated in vitro with ALV003 or placebo on patients with celiac disease with minimal exposure to the active treatment. This study was classified as a phase 0 trial because the patients were not exposed to active drug, but rather only to minute doses of heat-inactivated study treatment. ALV003-0801 demonstrated the following: ALV003-treated gluten was safely administered to HLA DQ2 celiac disease patients in a gluten challenge study. Gluten-specific T cell responses were abolished by ALV003 pre-treatment. No difference in diary reported symptoms between patients receiving ALV003 treated and placebo treated gluten challenge were observed.

### Example 2

ALV003-0811

Study ALV003-0811 titled: A Phase 1, Two Stage, Single Dose, Single-Blind, Placebo Controlled, Dose Escalation, Crossover Study of the Safety and Tolerability of ALV003 in Healthy Adult Volunteers and Subjects with Well-Controlled Celiac Disease, was run in the U.S. This was a First-in-Human study, where ALV003 was administered via a nasogastric tube in the form of a solution to subjects in the fasted state. Gastric samples were extracted from the stomach at pre-dose and at 16 time points post-dose out to 3 hours. Gastric samples were evaluated for the presence of ALV001 and ALV002 and their activity. These samples were also assessed ex vivo for the ability of ALV003 to digest gluten. This was a 2-stage protocol with healthy human volunteers being dosed in the first stage, and celiac disease patients dosed in the second stage. Twenty-four subjects (healthy human volunteers) were dosed with ALV003 (6 subjects at 100 mg, 6 subjects at 300 mg, 6 subjects at 900 mg, 6 subjects at 1800 mg). Four celiac disease patients were subsequently dosed with ALV003 at 300 mg. There were no serious adverse events reported in this study.

The ALV003-0811 trial demonstrated the following: single doses of ALV003 appeared to be safe and well tolerated at dose levels as high as 1800 mg in healthy fasting volunteers, and at a dose of 300 mg in celiac disease patients. There were no dose-limiting toxicities observed. Most adverse events were mild or moderate in intensity and self-limited. The most commonly reported adverse events were headache, dyspepsia, and nausea; no other adverse events were reported by more than one subject per dose group. There was a consistent dose-related increase in the presence of ALV003 components (activated ALV001 and ALV002) in the stomach in the fasted state. With increasing dose there was longer duration of presence of ALV003 (activated ALV001 and ALV002) in the stomach in the fasted state. The ALV003 that was detected in gastric fluid samples was active, both in its ability to cleave chromogenic substrates specific to ALV001 and ALV002, and in its ability to degrade gluten as measured by a gluten peptide ELISA. ALV003 and/or placebo caused a dose-dependent increase in intragastric pH. In the ALV003 group, this may have resulted in the persistence of ALV001 proenzyme at higher doses. ALV001 (active) and ALV002 were detected at all doses in the fasted stomach. Dose dependent ex-vivo degradation of gluten was seen in all doses tested. One subject who had a sustained elevation in gastric pH throughout the study had a detectable level of ALV001 in the plasma; no clinical or safety sequelae were noted. No subjects had detectable levels of ALV002.

### Example 3

ALV003-0812

Study ALV003-0812 titled: A Phase 1, Two Stage, Single-Blind, Single Dose, Placebo Controlled, Dose Escalation, Crossover Study of the Safety and Tolerability of ALV003 in Healthy Adult Volunteers and Subjects with Well-Controlled Celiac Disease following a Gluten-Containing Meal was run in the U.S. This study is similar to Study ALV003-0811 with the exception that study subjects received ALV003 along with a gluten-containing meal. The dose escalation scheme and study design were similar to study ALV003-0811. A total of 52 volunteers (healthy human volunteers) were dosed with ALV003 in this study; 14 subjects received 100 mg, 18 subjects received 300 mg, 14 subjects received 900 mg and 6 subjects received 1800 mg. One celiac disease patient was dosed with ALV003 at 300 mg. There were no serious adverse events reported in this study.

ALV003-0812 demonstrated the following: ALV003 degraded gluten in the stomach by 30 minutes when administered concomitantly with a standardized meal containing fat, protein and carbohydrate using an optimized sampling regimen. Doses of ALV003, as low as 100 and 300 mg, demonstrated biological activity. All 16 subjects dosed at these levels consistently yielded less gluten on gastric aspirates following exposure to ALV003 (mean gluten eliminated 78% vs. placebo; p<0.01,Wilcoxon Signed Rank test). The addition of sodium metabisulfite to the 300 mg dose group significantly increased the amount of gluten eliminated by ALV003 (p=0.021; Kruskal-Wallis test); addition of sodium metabisulfite effected no difference in clinical activity in the 100 mg dose group. ALV003 appeared to be safe and well tolerated up to doses of 1800 mg in healthy volunteers, as well as in the single celiac disease patient dosed at 300 mg. All adverse events reported in the trial were mild or moderate in intensity. The most commonly reported adverse events were headache, flatulence, nausea, dyspepsia and diarrhea. One subject showed an increase in anti-ALV001 antibody titers after dosing; no subjects had an increase in anti-ALV002 antibody titers after dosing. Of note, the antibodies used to detect ALV001 and ALV002 could not discriminate between antibody isotypes, nor could the antibodies distinguish between intact, full-length drug or drug fragments; however, there were no immunologic adverse events related to the study drug reported in any of the subjects who had a positive antibody response.

### Example 4

ALV003-0921

Study ALV003-0921 titled “A Phase 2a, Double-Blind, Placebo Controlled Study of the Efficacy, Safety and Tolerability of 6-weeks Treatment With ALV003 In Patients With Well-Controlled Celiac Disease” was conducted at Tampere University in Tampere, Finland and was designed to assess the ability of orally administered ALV003 to protect against gluten-induced mucosal injury.

Gluten ingestion is associated with changes in small intestinal mucosal morphological and immunological parameters as well as in serological parameters and clinical symptoms. However, these responses are not uniform in all study patients. The correlation between symptoms, celiac disease serology, and intestinal pathology differs in patients but all are gluten induced and sensitive outcomes. The small intestinal biopsy remains the gold standard for diagnosis of celiac disease. In this study, even short-term gluten ingestion could be correlated with changes in small bowel pathology, the primary outcome of the study, as well as in immunohistochemistry. Gluten ingestion is also associated with changes in intestinal pathology, particularly related to upregulation of certain early immunological markers [(Shiner 1972), (Korponay-Szabo 2004), (Kaukinen 2005)]. Gastroscopy associated with duodenal biopsy is likely to be sensitive at detecting gluten-related mucosal injury, and was therefore utilized in this study. Biopsy samples were morphometrically evaluated by a blinded reader for both architectural changes and immune-mediated injury in the Coeliac Disease Study Group laboratory at the University of Tampere, Tampere Finland.

Patients were required to be previously diagnosed by intestinal biopsy and well-controlled on a gluten-free diet for at least 1 year and have negative celiac disease-specific serologies at baseline. Following screening and baseline duodenal biopsy, patients were randomized 1:1 to either ALV003 or placebo (study treatment) three times a day (at each of the major meals) along with a defined gluten challenge (see below) at each meal for up to 42 days. Patients then underwent a follow up duodenal biopsy and were followed for another 4 weeks for safety assessments.

The primary endpoint for the study was the change from baseline to post-treatment villus height to crypt depth (Vh:Cd) ratio. Additionally, changes in the density of intraepithelial lymphocytes and IgA deposits on TG2 in the villi were measured. Also measured were changes in serologic markers of celiac disease, including, IgA class TG2 antibodies, IgA and IgG class deamidated gliadin peptide antibodies, and serum IgA class endomysial antibodies.

The study was conducted in three stages: in Stage 1 patients received a 2 gram TID (three times per day) gluten challenge; in Stage 2, patients received a 1 gram TID gluten challenge; and in Stage 3, patients received a 0.5 gram TID gluten challenge. Seventy four patients were randomized to treatment with either ALV003 or placebo treatment for 6 weeks. Patients were instructed to continue on their normal gluten-free diet during the course of the study.

In Stage 1, 33 patients were enrolled and 7 patients terminated the study early: three because of gastrointestinal adverse events assessed to be induced by gluten and four for medical reasons unrelated to either the study treatment or ingestion of gluten (i.e., upper respiratory infection, eczema, flu-syndrome with syncope, and a sick-sinus syndrome in another necessitating placement of a cardiac pacemaker); there were two serious adverse events (SAEs) in one patient (atrial fibrillation and sick sinus syndrome) that were considered to be not related to the study treatment. In Stage 2, 31 patients were enrolled, two terminated the study early due to adverse events assessed as related to gluten ingestion; there were no SAEs. In Stage 3, 10 patients were enrolled and one patient terminated participation at 4 weeks due to a gluten associated gastrointestinal adverse event; there were no serious adverse events.

The dose of gluten initially used in this ALV003-0921 study elicited both symptomatic and immunologic responses in patients with celiac disease. Intestinal mucosal data from the Tampere University Celiac Research Service Laboratory on the patients receiving 2 g gluten and 300 mg ALV003 given three times daily revealed that the proportion of patients showing change in Vh:Cd ratio exceeded expectations. The original study design was based on the assumption that approximately 50% of the patients in the placebo group would show Vh:Cd changes of >0.5 units. The actual data from these patients revealed that over 80% of patients showed changes of this magnitude.

For patients randomized into the second stage of this ALV003-0921 study, the amount of gluten was reduced from 6 g daily (given as 2 g three times daily) to 3 g daily (given as 1 g three times daily). Intestinal mucosal data on these patients revealed that over 75% of patients showed Vh:Cd changes of ≥0.5 units. The amount of gluten was further reduced to 1.5 g daily (given as 0.5 g three times daily) for patients enrolled in the third stage of this ALV003-0921 study. Intestinal mucosal data on 10 patients in stage 3 of this study revealed that over 70% of these patients showed Vh:Cd changes of ≥0.5 units.

The differences in the primary and secondary endpoints between the ALV003- and placebo-treated groups were not statistically significant. Careful analysis of the available data suggested that the dose of gluten administered exceeded the extent of predicted mucosal injury, and that ALV003 given in the manner administered in this study failed to mitigate gluten-induced mucosal damage sufficiently to demonstrate statistically significant results. However, a clear dose-response curve was generated that demonstrated quantifiable differences in the extent of mucosal injury in proportion to the amount of gluten ingested daily over six weeks.

The adverse event profile of the patients enrolled in the ALV003-0921 study was believed to be primarily related to the daily amount of gluten ingested. Specifically, the number of adverse events, their severity and the number of patients who discontinued study treatment appeared to be directly related to the amount of gluten ingested per day. Initial review of the data collected in the ALV003-0921 study suggested that there was a dose response trend between the dose of the gluten challenge and the extent of mucosal injury. response trend between the dose of the gluten challenge and the extent of mucosal injury. In contrast, there was not to a clear signal of ALV003 activity. There were several possible explanations considered for the observed data, including one or more of the following: ALV003 instability in the gastric compartment; inadequate mixing of ALV003 and gluten in the gastric compartment; inadequate ALV003 enzymatic activity; or difficulty of patient adherence to dosing regimen. Results from Study ALV003-1021 (see below) demonstrated that addition of an antioxidant (e.g. 100 mg of cysteine) in the formulation resulted in better efficacy, indicating other explanations may not have been a primary factor in the efficacy observed.

### Example 5

ALV003-1021

The study described in this example was designed as a phase 2a, double-blind, placebo-controlled trial in celiac disease patients to demonstrate the ability of ALV003 to inhibit gluten-induced ill health using a study design of 6-weeks challenge with gluten. At the same time, the safety and tolerability of the study treatment was studied. Celiac disease patients (meeting entry criteria) with well controlled disease were enrolled into the study.

Study Design

Study Population

The study population consisted of patients between the ages of 18 and 75 with well-controlled celiac disease. In particular, the target population for this study was patients with celiac disease who had well-controlled disease without significant co-morbidities and had been adherent to a gluten-free diet for at least 1 year prior to study entry. Such patients were enrolled in the study because they were likely to have a normalized serology and small-intestinal mucosa, and thus changes caused by gluten challenge could be more readily detected.

Inclusion Criteria

Patients were biopsy-proven celiac disease patients in otherwise good health that met the following conditions: age 18 to 75; history of biopsy-positive celiac disease (hospital records or the Social Insurance Institution of Finland, KELA, issued certificate); adherence to a gluten-free diet for at least 12 months prior to randomization (documented by medical history); TG2 antibody negative tested using the rapid point-of-care finger tip whole blood test (Biocard™ Celiac Test, AniBiotech Ltd); no history of acute illness for the past 4 weeks; willing to consume a large meal (eg, dinner) each day, adhere to a 6-week gluten challenge and undergo 2 on-study upper gastrointestinal endoscopies including multiple biopsies; and signed informed consent.

Exclusion Criteria

Patients were excluded if they meet any of the following exclusion criteria: active dermatitis herpetiformis lesions at the time of screening; history of IgE-mediated reactions to gluten; any clinical contraindications to performing an endoscopy with intestinal biopsy; received any systemic biologics (such as monoclonal antibodies or other protein therapeutics where the half life overlaps with study start) within 6 months prior to study start; taking any oral probiotic supplements (not including probiotics contained in commercially available food preparations) 6 months prior to entry; use of any immunosuppressive medications (i.e., for chronic treatment of autoimmune disease or transplant-rejection prophylaxis) 6 months prior to entry; use of systemic cortisone-like medications within 28-days prior to and during study treatment; history of alcohol abuse or illegal drug use (e.g. amphetamines, barbiturates, benzodiazepines, cannabinoids, cocaine, and opiates) within the past 12 months; laboratory values: elevated liver function tests (ALT, AST, Alk Phos and GGT >2.5 times the upper limit of normal (ULN); bilirubin >1.5 ULN; serum creatinine >1.5 ULN; hemoglobin <10 g/dL or 100 g/L; calcium <2.0 mmol/L; platelet count <75.0×10⁹/L or 75,000/mm³; partial thromboplastin time (PTT) or prothrombin time (PT/INR) >1.5 ULN; serum potassium <3.0 mmol/L, >5 mmol/L; total white blood cell count (WBC)<3.0×10⁹/L or 3000/mm³; and total lymphocyte <1.0×10⁹/L or 1000/mm³; current untreated or active peptic ulcer disease, Grade B or greater esophagitis, motility disorders such as irritable bowel syndrome, functional dyspepsia, inflammatory bowel disease, and symptomatic GERD (gastroesophageal reflux disease) other than celiac disease; positive pregnancy test at screening and just prior to gluten challenge; unwilling to practice highly effective birth control (unless surgically sterilized or post-menopausal); other than oral contraceptives, use of prescribed medications or over-the-counter medications that in the opinion of the investigator might interfere with study results; current use of anticoagulants (warfarin sodium, heparin, full-dose aspirin [325-650 mg/dose] or clopidogrel) during the 7-day period prior to randomization; received any experimental drug within 14 days of randomization; in the case of experimental biologics at least 6 months prior to randomization; the existence of any uncontrolled chronic disease or condition [for example HIV-AIDS, hepatitis, Type 1 or 2 diabetes, or cancer (other than skin cancer)], other than celiac disease; uncontrolled complications of celiac disease, which in the opinion of the PI could affect immune response, or pose an increased risk to the patient (e.g. Type 1 diabetes or other autoimmune disease); known allergy or hypersensitivity to any of the components of the placebo, ALV003, E. coli or E. coli-derived proteins; known adverse respiratory effects caused by sulfites; and any medical condition, other than celiac disease, which, in the opinion of the study investigator could adversely affect the patient's participation in the trial.

Primary Efficacy Endpoint

The primary efficacy endpoint of this demonstration was change in intestinal villus morphology as determined by changes in villus height to crypt depth ratio from baseline to end of treatment in the ALV003 vs. the placebo group.

Secondary Efficacy Endpoints

Additional outcomes evaluated were effects on symptoms, celiac disease serology and small bowel mucosal inflammatory changes. Thus, secondary efficacy endpoints included change from baseline to end of treatment of: (i) measures of small intestinal mucosal inflammation: (a) density of intraepithelial lymphocytes; and (b) IgA deposits on TG2; and (ii) measures of serologic markers of celiac disease: (a) IgA class TG2 antibodies; (b) IgA and IgG class deamidated gliadin peptide antibodies; and (c) serum IgA class endomysial antibodies.

Exploratory Endpoints

Exploratory endpoints included change from baseline to end of treatment in: (i) measures of celiac disease related symptoms and quality of life: (a) GSRS, CDQ and SF-36v2 questionnaires; (b) stool diary; and (c) celiac disease-related VAS; and (ii) measures of malabsorption in blood (hemoglobin, erythrocyte folate, calcium).

Primary Safety Endpoint

Primary safety endpoints were safety and tolerability of ALV003, pharmacokinetics of ALV003, and antibody response to ALV003. The principal safety outcome measures were incidence of Adverse Events (AEs) including clinically significant laboratory evaluations and Serious Adverse Events (SAEs).

Adverse Events

An adverse event was defined as any untoward medical occurrence (e.g., sign, symptom, disease, syndrome, intercurrent illness) that occurred in a study patient once they had been enrolled in the study, regardless of the suspected cause. Severity referred to the intensity of a specific event. There were three levels of severity, defined as follows: Mild: Noticeable, but does not disrupt normal daily activity; Moderate: Sufficient to reduce or disturb normal daily activity; and Severe: Incapacitating, significantly interferes with or prevents normal daily activity.

Treatment

All patients were screened to meet entry criteria within 28 days of beginning treatment. Once patient entry criteria were met, patients were randomized to receive active or placebo treatment along with a gluten challenge for 6-weeks.

Cysteine was added to the drug product to enhance ALV003 stability (cysteine was added to the placebo treatment as well).

In Stage 1, mixing of ALV003 with gluten was performed before ingestion. In Stage 2, ALV003 and gluten were administered separately to demonstrate the effectiveness of mixing of ALV003 and gluten in the stomach.

In order to simplify the study treatment regimen and decrease the patient-to-patient variability, the daily gluten dose in both stages of the study was given once daily along with a total daily dose of 900 mg ALV003.

In Stage 1, baseline biopsy data showed that in the placebo treated patients, the mean Vh:Cd was 3.0, and at the 6 week follow up biopsy, was 2.4, for a change in Vh:Cd of −0.6. The magnitude of the absolute change in Vh:Cd was relatively small; therefore an increase in the gluten dose to 2 grams/day in Stage 2 was deemed likely to increase the change in Vh:Cd to a level that would be considered more clinically relevant as well as increase the likelihood of having a sufficient signal and meaningful event rate in the placebo arm. Additionally, careful evaluation of the patients from the ALV003-0921 study suggested that even with an increase in gluten dose to 2 grams, would have a minimal effect on the number and severity of gluten-related adverse events. Finally, by increasing the dose of the gluten challenge to 2 grams/day, (coupled with an increase in the sample size of the study), the chances of showing a statistically significant difference between the two treatment groups were improved.

The following table summarizes and compares treatment in the two stages of the study.

| | ALV003/Placebo | Gluten | Fre- | Form of Gluten |
|---|---|---|---|---|
| Stage | Dose | Dose | quency | Administration |
| 1 | 900 mg | 1.5 g | QD | Bread crumbs suspended in |
| | | | | liquid, mixed with study |
| | | | | treatment and ingested |
| | | | | immediately |
| 2 | 900 mg | 2 g | QD | Bread crumbs suspended in |
| | | | | liquid, ingested separately |
| | | | | from study treatment |

Patients were instructed to consume the drug product (or placebo) as follows: (i) pour room temperature Mehukatti juice (commercially marketed in Finland) to the top of the label (about 125 mL) of ALV001 dose/bottle (or placebo) and swirl gently to dissolve powder; pour room temperature Mehukatti juice to the top of the label of breadcrumbs (about 125 mL) and swirl gently to mix; pour room temperature Mehukatti juice to the top of the label (about 60 mL) of ALV002 dose/bottle (or placebo) and swirl gently to mix powder (the patients were advised that not all powder would dissolve and the mixture might appear cloudy); the patients were advised to eat about half of their meal, and then to swirl gently and drink all of ALV001 (or placebo), swirl gently and drink all of breadcrumbs; add more Mehukatti juice to breadcrumbs, swirl gently and drink to rinse off remaining breadcrumbs; and swirl gently and drink all of ALV002 (or placebo). The order of dissolving and administering ALV001 and ALV002 was varied for some administrations.

Patients were instructed to return to the clinic 1 week after initiation of treatment (Visit 3). Patients were contacted by the clinical study site staff by telephone 2 and 4 weeks after initiation of study treatment (Visits 4 and 5) and instructed to return to the clinic 6 and 10 weeks after initiation of treatment (Visits 6 and 7). Safety was evaluated throughout the study. Intestinal biopsies were obtained for all patients prior to receiving active treatment (but the baseline biopsy was not used for screening purposes), and after completion of the 6-week treatment. Any patients who dropped out beyond at least seven days of study treatment during the 6-week gluten exposure period due to clinical symptoms were encouraged to undergo an esophago-gastro-duodenoscopy (EGD) examination and small intestinal biopsy to determine whether gluten-induced mucosal changes from baseline were likely to have been the cause of the clinical symptoms.

At Visit 2, patients were provided with a study treatment kit containing enough study treatment for 1 week. Patients received a kit containing a 5 week supply of study treatment at Visit 3. If the patient required additional study medication to meet the visit schedule, additional bottles were distributed and documented by the study nurse. Patients were contacted by the clinical study site staff at Visits 4 and 5 to assess safety (e.g., eliciting reporting of any adverse events) and check on patient study medication compliance. Patients were instructed to retain all used study bottles and to return the kits to the clinic at each visit. Patients were asked to complete a meal/dosing diary. Upon return to the clinic, all of the bottles were counted. During study visits and telephone calls for Visits 4 and 5 the meal/dosing diary was assessed for compliance with the required regimen; any missed or irregular dosing was noted in the patient's chart. Patient non-compliance with the dosing regimen was recorded; patients were reminded of compliance requirements at each study visit and telephone call.

The patients were instructed to self-administer a foodstuff containing a specified amount of gluten along with the study treatment once a day with dinner. The patients otherwise followed their normal gluten-free diet. The meal/dosing diary was used to track the meal, study treatment and the gluten intake throughout the study. Compliance with gluten and dosing was monitored at clinic visits and at the Visit 4 and Visit 5 telephone calls. Patient non-compliance with the challenge regimen was recorded; patients were reminded of compliance requirements at each study visit.

Tests and Evaluations

Celiac serology consisted of measurement of IgA class TG2 antibodies measured by an enzyme-linked immunosorbent assay (ELISA, QUANTA Lite™ h-tTGIgA, INOVA Diagnostics, Inc.); blended IgA and IgG class deaminated gliadin peptide antibodies by an ELISA method (QUANTA Lite™ Celiac DGP Screen, INOVA Diagnostics, in order to detect antibody titer changes in potential IgA deficient individuals); and serum IgA class endomysial antibodies, measured using an indirect immunofluorescence method with human umbilical cord as antigen [(Sulkanen, Halttunen et al. 1998), (Stern 2000)]. The whole blood finger tip celiac antibody point of care test was performed at patient screening (Biocard™ Celiac Test, AniBiotech) (Raivio 2006).

Upper gastrointestinal endoscopy and biopsy was performed at an accredited facility by qualified, experienced, endoscopists. Standard procedures were followed for sedation, gastroscopy and biopsy. An EGD was performed using a video gastroscope at baseline (up to 3 days before gluten challenge) and at end of the 6-week study (within 3 days after ending gluten challenge). All possibly significant macroscopic abnormalities in the esophagus, stomach, duodenal bulb and descending duodenum were documented, and routine biopsy samples for histopathology assessment were taken as necessary.

Approximately seven to ten small-bowel biopsy specimens were taken from the distal part of the duodenum; approximately three of which were formalin-fixed, paraffin embedded, stained with hematoxylin-eosin, and studied under light microscopy. Approximately three samples were snap-frozen and studied immunohistochemically.

The primary clinical efficacy assessment was performed using the formalin-fixed hematoxylin-eosin stained specimens. Morphometric analysis of the small bowel biopsy specimens involved measurement of villus heights (Vh) and crypt depths (Cd) and calculation of their ratios (i.e., Vh:Cd). These measurements were performed at multiple locations in order not to miss patchy forms of villous atrophy. Vh:Cd was calculated as a mean of 3-5 villus-crypt units. If mucosal patchiness was observed, results were calculated from the most severely damaged area. These Vh:Cd measurements were performed on both baseline EGD specimens (before gluten exposure) and from specimens at the end of gluten exposure. The villus height and crypt depth measurements were obtained from well-oriented biopsy samples as previously described [(Kuitunen P 1982), (Holm, Mäki et al. 1992), (Kaukinen, Collin et al. 1999), (Peraaho, 2003), (Holm, Mäki et al. 2006), (Salmi 2006)]. Poorly oriented sections were discarded and, when necessary, the samples were dissected repeatedly until they were of good quality.

Approximately three samples were snap-frozen for immunohistochemical staining to perform secondary efficacy assessments. The specimens were freshly embedded in optimal temperature compound (Tissue-Tek, Miles Inc.), snap-frozen in liquid nitrogen, and stored at −70° C. Immunohistochemical studies were carried out on 5 μm-thick frozen sections. CD3+ IELs were stained with monoclonal antibody Leu-4 (Becton Dickinson), αβ+ IELs with monoclonal PH antibody (Endogen) and γδ+ IELs with TCRγ antibody (Endogen). IELs were counted with a 100× flat-field light microscope objective in randomly selected surface epithelium; at least 30 fields of 1.6 mm epithelial length are counted and the density of IELs expressed as cells/millimeter of epithelium [(Mäki 1991), (Holm, Mäki et al. 1992), (Iltanen, Holm et al. 1999), (Kaukinen, Collin et al. 1999), (Kaukinen 2001), (Jarvinen, Kaukinen et al. 2003), (Peraaho, Kaukinen et al. 2003), (Kaukinen 2005), (Salmi 2006)].

Small intestinal mucosal TG2-specific IgA deposits were studied by direct and indirect immunofluorescence methods from frozen sections and graded 0-3 as to their intensity as previously described [(Shiner and Ballard 1972), (Korponay-Szabo 2004), (Kaukinen 2005), (Salmi 2006)]. Specifically, three unfixed, 5-μm-thick sections from frozen small bowel specimens were investigated for IgA deposits by direct immunofluorescence using fluorescein isothiocyanate-labelled rabbit antibody against human IgA (Dako A S). In untreated celiac disease a clear gluten-induced subepithelial IgA deposition can be found along the villus and crypt epithelium and around mucosal vessels. This deposition disappears on gluten-free diet and appears again upon gluten exposure. To confirm that celiac-type IgA deposits co-localize with TG2, three sections were stained for human IgA and by indirect immunofluorescence for TG2 (monoclonal mouse antibody CUB7402, NeoMarkers, followed by rhodamine-conjugated anti-mouse immunoglobulin antibodies, Dako AS).

For patients with a history of DH, representative baseline diagrams were drawn and photographed, if possible, of historic lesion locations. For patients who developed a rash during the course of study treatment, diagrams were drawn reporting the rash area locations and, if possible, photographs were taken of the specific areas of involvement.

The patient questionnaires were the Gastrointestinal Symptom Rating Scale (GSRS), CDQ and the SF-36v2™ Health Survey (SF-36). The clinical study site provided two GSRS, CDQ and celiac disease-related VAS to the patients at Visit 3. At Visits 4 and 5, the clinical study site contacted the patient by telephone and reminded her/him to complete the GSRS, CDQ and celiac disease-related VAS. The patients were instructed return the completed questionnaires and celiac disease-related VAS to the clinical study site at Visit 6.

The GSRS was used to assess gastrointestinal symptoms. The score is comprised of 15 items with five sub-dimensions, diarrhea, indigestion, constipation, abdominal pain and reflux. Rating is based on a seven-point Likert scale where higher scores indicate more severe gastrointestinal symptoms [(Svedlund, Sjodin 1988), (Dimenas, 1993), (Lohiniemi 1998), (Mustalahti, Lohiniemi et al. 2002), (Viljamaa, Collin et al. 2005)].

The CDQ, a disease-specific, health-related quality of life measure for adults with Celiac Disease, consists of four scales (gastrointestinal symptoms, emotional well-being, social restrictions and disease-related worries) with seven items on each scale using a 7-point Likert scale for scoring [(Hauser 2007), (Hauser 2007)]. The SF-36 questionnaire is used to assess health-related quality of life [(McHorney, Ware et al. 1994), (Hallert 1998), (O'Leary 2004)]. The raw score on all 36 items are re-scored from 0 to 100, higher scores indicating better health and quality of life. Items are then divided into eight sub-dimensions: physical functioning, role limitations due to physical problems, bodily pain, general health, vitality, social functioning, role limitations due to emotional problems, and mental health. SF-36v2 yields a score for each of these health domains, as well as summary scores for both physical and mental health and a single health utility index.

A celiac disease-specific VAS was used to score gluten-induced ill health. This comprised a 100 mm line where 0 mm depicts “excellent health, no symptoms and signs of celiac disease” and 100 mm depicts “poor health, very severe symptoms and signs of celiac disease”. Patients marked their status on the visual analog scale at Visits 1, 3, 5 and 7. The position of the mark was measured in millimeters and ALV003 and placebo were compared.

Patients also monitored their stool output number and fecal consistency (hard lumps, normal, loose, watery) daily over the course of the study and completed a stool diary with their observations. Patients were instructed as to what minimally constitutes a full meal. Patients were required to confirm that each dose was taken with a meal in accordance with the specific study stage dosing instructions. Adherence to this was tracked by the patient via a meal/dosing diary. Patients received ALV003 or placebo and a gluten foodstuff with a meal for 6 weeks.

Collection of Adverse Events began after the initiation of study treatment and continued throughout treatment.

Data Analysis

The Safety Population included all patients who received study treatment. The Evaluable Population included all randomized patients who received at least 7 days of study treatment and had a post-treatment intestinal biopsy. This population was used for the primary efficacy analysis of the data from each stage of the study, as well as for all secondary and exploratory efficacy analyses in all stages of the study.

In each stage the data generated were analyzed to assess the safety and appropriateness of the gluten challenge dose by assessing the effect of the gluten challenge on the morphology of the small intestinal mucosa. The interim evaluation after Stage 1 allowed for real-time safety monitoring and identification of any study design or operational issues to be addressed prior to initiation of Stage 2 of the study, thus increasing the likelihood that the outcome of the subsequent stage of the study would provide a definitive demonstration of efficacy.

All secondary and exploratory efficacy endpoints were analyzed separately for each stage of the study in the evaluable population from each stage. The distribution of changes from baseline in the two groups were compared using the Wilcoxon-Mann-Whitney test, and in some cases where the distribution of the data were normally distributed, a 2-sample, 2-sided t-test at 5% level. Analyses were based on available data; as such, no imputation was performed for missing data.

Results

Stage 1: Preliminary, interim assessment of the data following completion of enrollment of Stage 1 (15 women and 5 men) revealed the following.

The Vh:Cd change from baseline to 6 weeks in the placebo treated group was approximately −0.6 but in the ALV003 treated group was approximately −0.2; the pooled standard deviation of the primary endpoint was empirically demonstrated to be 0.51. The proportion of patients showing either no change or positive change in their Vh:Cd ratios was 0/8 in the placebo group vs. 5/9 in the ALV003 treatment group (p=0.029). The changes in IEL number (both CD3+ and αβ+ T cells) showed a statistically significant difference between the treatment groups (p=0.02), suggesting that protection was being conferred by ALV003 from gluten-associated lymphocytic infiltrates. There were positive trends (not statistically significant) in TG2 antibody titers and DGP antibody titers (serologies) in favor of the ALV003 treatment group. No obvious treatment-related adverse events and no SAEs were observed. Two patients withdrew early from the study due to gastrointestinal adverse events; the principal investigator attributed these adverse events to the gluten challenge.

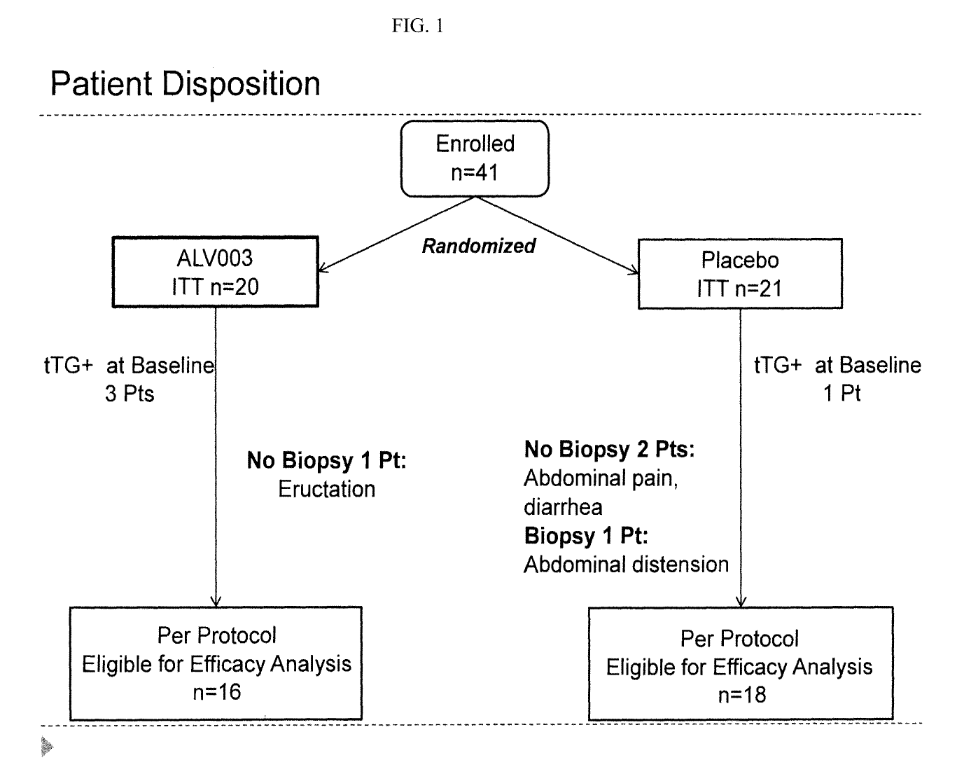

Stage 2: The patient disposition in the treatment and placebo groups is shown in FIG. 1. Sixteen patients in the treatment arm and 18 patients in the placebo arm were eligible for efficacy analysis.

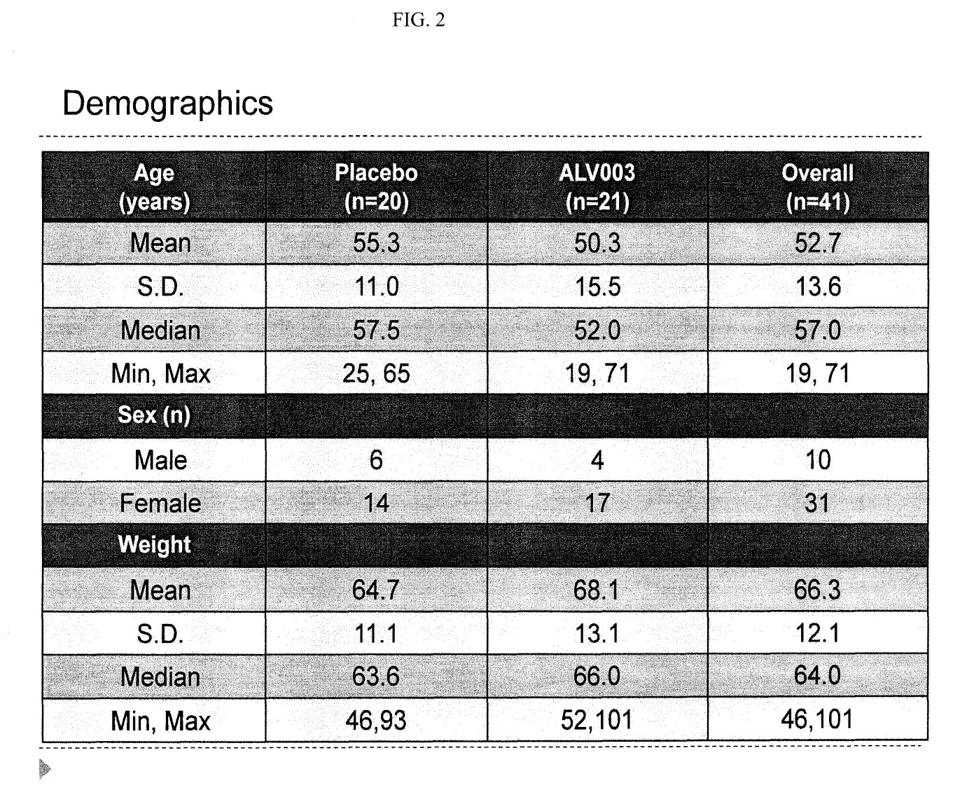

The demographics of the patients are presented in FIG. 2.

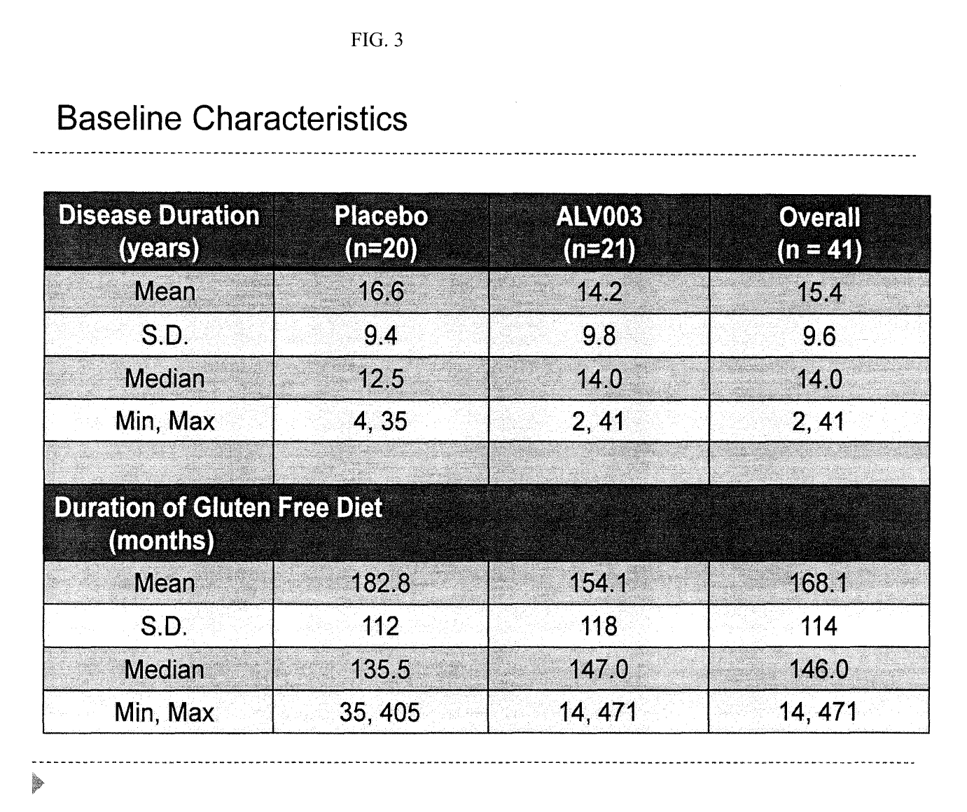

The baseline characteristics of the study population are shown in FIG. 3.

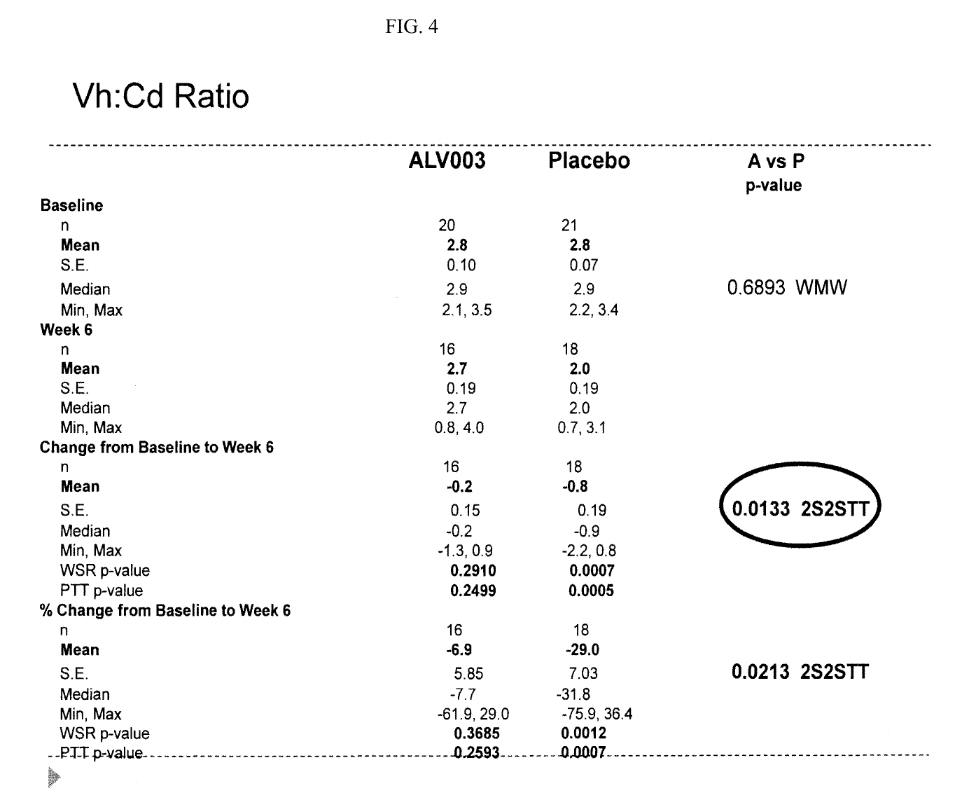

The Vh:Cd ratios are shown in FIG. 4.

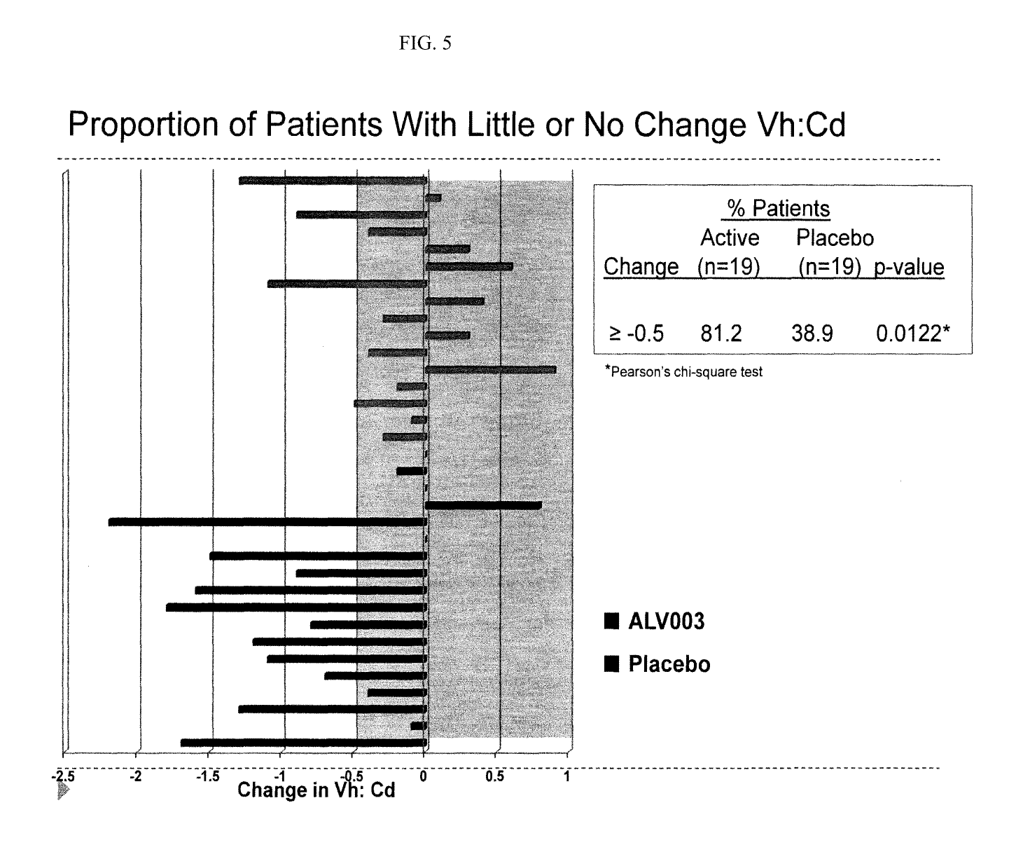

The percent change from baseline was of primary interest; the proportion of patients with little or no change in the Vh:Cd ratio is shown in FIG. 5.

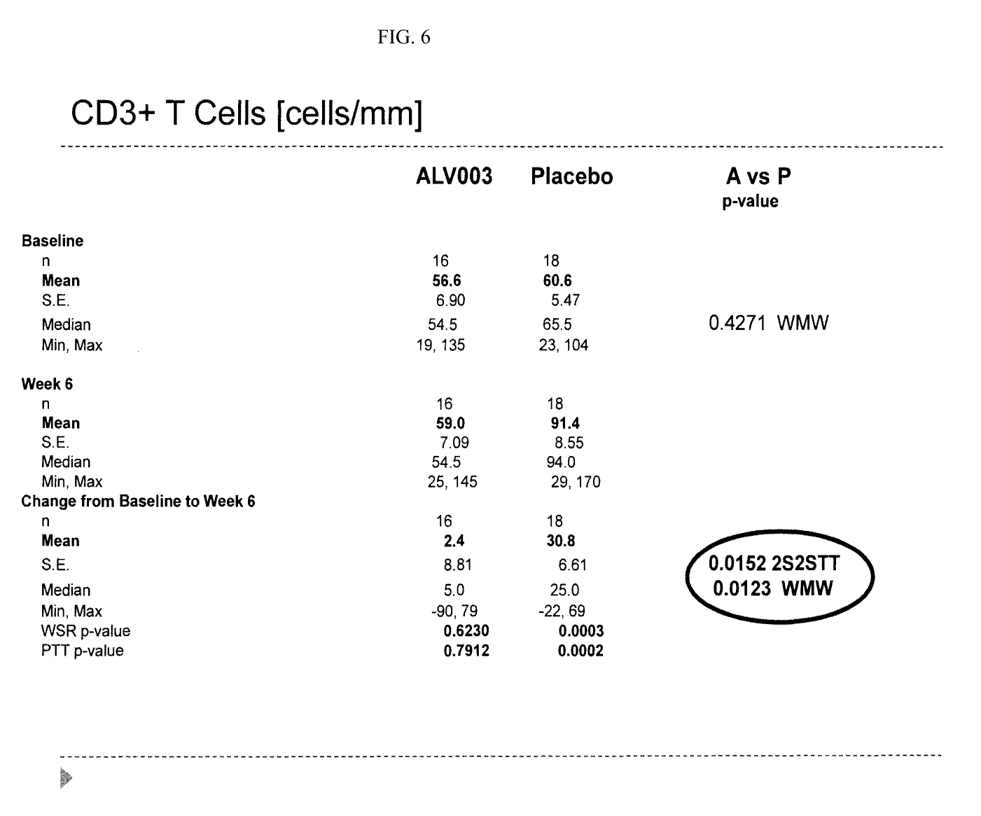

The changes in the amount of CD3+ T cells in the treatment and placebo groups are shown in FIG. 6. The treatment resulted in a dramatic drop in the number of CD3+ T cells/mm epithelium in patients treated with ALV003.

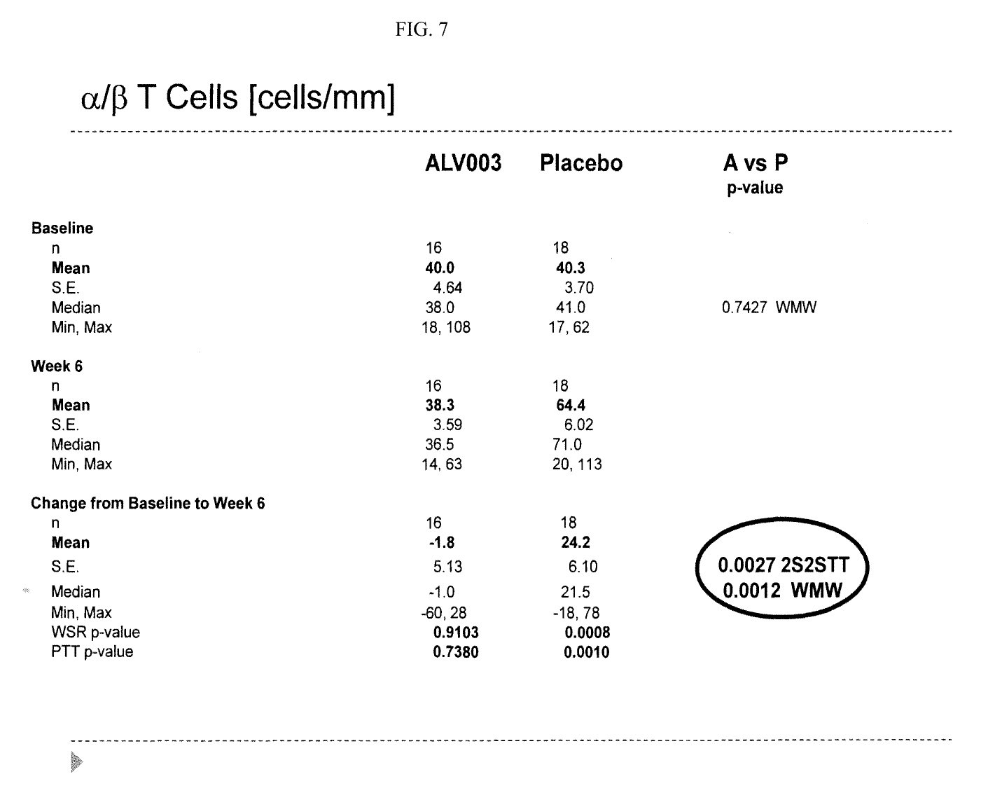

The changes in the amount of αβ T cells in the treatment and placebo groups are shown in FIG. 7. The treatment resulted in a dramatic drop in the amount of αβ T cells in patients treated with ALV003.

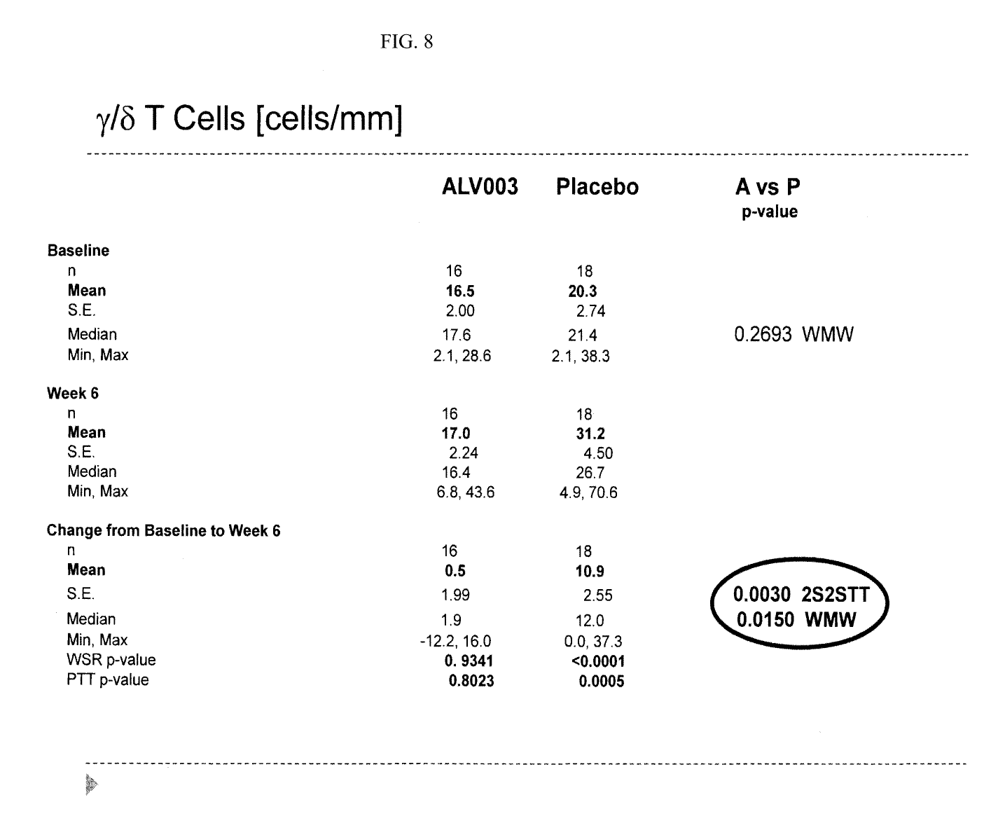

The changes in the amount of γδ T cells in the treatment and placebo groups are shown in FIG. 8. The treatment resulted in a dramatic drop in the amount of γδ T cells in patients treated with ALV003.

A summary of the mean morphometric changes from Stage 2 are shown in the following Table.

| | | | Delta (Wk 6) − (Wk 0) | | |
|---|---|---|---|---|---|
| | Mean Baseline Value | | ALV003 | Placebo | |
| | ALV003 | Placebo | N = 16 | N = 18 | p value* |
| Vh:Cd | 2.8 | 2.8 | −0.18 | −0.82 | 0.013 |
| CD3 IELs | 57 | 61 | +2.4 | +30.8 | 0.015 |
| αβ IELs | 40 | 40 | −1.8 | +24.2 | 0.003 |
| γδ IELs | 17 | 20 | +0.5 | +10.9 | 0.003 |

*2-sample, 2-sided t-test

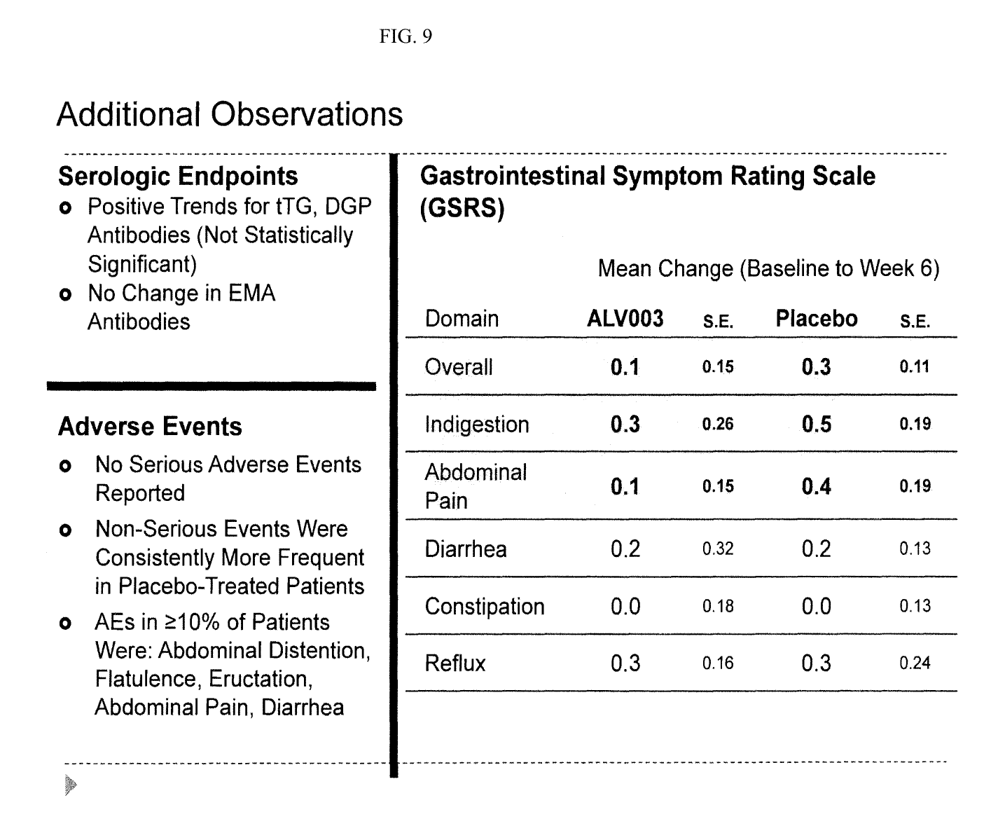

Upon completion of the study, no serious adverse events were reported. Adverse events in at least 10% of the patients were abdominal distention, flatulence, eructation, abdominal pain, nausea, headache, fatigue and diarrhea. A summary of these observations in presented in FIG. 9.

Discussion

The multiple lines of evidence, when taken together, demonstrated that the observed protection of the intestinal mucosa against gluten-induced injury was highly unlikely to be due to random chance. There were several inferences drawn from the data. First, the experimental methodology used in Stage 1 succeeded in demonstrating that digestion of the proline- and glutamine-rich gluten peptides could obviate injury to the mucosa. Second, control for mixing of enzyme and substrate appeared to be important. Third, addition of cysteine to the formulation (to increase stability of ALV003) appeared to impart beneficial properties to the formulation.

The stage 2 data demonstrated, in a clinically-relevant setting, the ability of ALV003 to protect celiac disease patients from gluten-induced mucosal injury. In this rigorously controlled clinical trial, in the context of everyday gluten-free meals, statistically significant differences in villus morphometric responses to daily gluten challenges were observed between the treatment and placebo groups. Although the study size did not allow demonstration of statistically significant differences in serologies and symptoms, the direction of the data trends was consistent with the morphometric changes in the villi.

For the first time in medical history, a pharmaceutical agent targeting degradation of proline- and glutamine-rich gluten peptides, has been shown to have the ability to attenuate gluten-induced injury to the mucosa in celiac disease patients. Accordingly, the study proves that targeting proline- and glutamine-rich gluten peptides is a viable approach for the treatment of celiac disease.

### REFERENCES

Abdulkarim, A. S., L. J. Burgart, et al. (2002). “Etiology of nonresponsive celiac disease: results of a systematic approach.” Am J Gastroenterol 97(8): 2016-21.
Anderson, R. P., P. Degano, et al. (2000). “In vivo antigen challenge in celiac disease identifies a single transglutaminase-modified peptide as the dominant A-gliadin T-cell epitope.” Nat Med 6(3): 337-42.
Anderson, R. P., D. A. van Heel, et al. (2006). “Antagonists and non-toxic variants of the dominant wheat gliadin T cell epitope in coeliac disease.” Gut 55(4): 485-91.
Bardella, M. T., P. Velio, et al. (2007). “Coeliac disease: a histological follow-up study.” Histopathology 50(4): 465-471.
Bethune, M. T., P. Strop, et al. (2006). “Heterologous expression, purification, refolding, and structural-functional characterization of EP-B2, a self-activating barley cysteine endoprotease.” Chem Biol 13(6): 637-47.
Bingley, P. J., A. J. K. Williams, et al. (2004). “Undiagnosed coeliac disease at age seven: population based prospective birth cohort study.” BMJ 328(7435): 322-323.
Catassi, C., E. Fabiani, et al. (2007). “A prospective, double-blind, placebo-controlled trial to establish a safe gluten threshold for patients with celiac disease.” Am J Clin Nutr 85(1): 160-6.
Catassi, C., M. Rossini, et al. (1993). “Dose dependent effects of protracted ingestion of small amounts of gliadin in coeliac disease children: a clinical and jejunal morphometric study.” Gut 34(11): 1515-9.
Cerf-Bensussan, N., T. Matysiak-Budnik, et al. (2007). “Oral proteases: a new approach to managing coeliac disease.” Gut 56(2): 157-60.
Ciclitira, P. (1984). “Clinical testing of gliadin fractions in coeliac patients.” Clin Sci 66(3): 357-64.
Collin, G. (2004). “Complete small intestinal mucosal recovery is obtainable in the treatment of celiac disease.” Gastrointestinal Endoscopy 59(1): 158.
Collin, P. (2005). “Antiendomysial and antihuman recombinant tissue transglutaminase antibodies in the diagnosis of coeliac disease: a biopsy-proven European multicentre study.” Eur J Gastroenterol Hepatol 17(1): 85-91.
Collin, P., L. Thorell, et al. (2004). “The safe threshold for gluten contamination in gluten-free products. Can trace amounts be accepted in the treatment of coeliac disease?” Alimentary Pharmacology & Therapeutics 19(12): 1277-1283.
Corrao, G., G. R. Corazza, et al. (2001). “Mortality in patients with coeliac disease and their relatives: a cohort study.” The Lancet 358(9279): 356-361.
Dieterich, W. (1998). “Autoantibodies to Tissue Transglutaminase as Predictors of Celiac Disease.” Gastroenterology 115: 1317-1321.
Dimenas, E. (1993). “Methodological aspects of evaluation of Quality of Life in upper gastrointestinal diseases.” Scand J Gastroenterol Suppl 199: 18-21.
Farrell, R. J. and C. P. Kelly (2002). “Celiac Sprue.” N Engl J Med 346(3): 180-188.
Fasano, A., I. Berti, et al. (2003). “Prevalence of celiac disease in at-risk and not-at-risk groups in the United States: a large multicenter study.” Arch Intern Med 163(3): 286-92.
Fasano, A. and C. Catassi (2001). “Current approaches to diagnosis and treatment of celiac disease: an evolving spectrum.” Gastroenterology 120(3): 636-51.
FDA (2006). “Guidance for Industry Patient-Reported Outcome Measures: Use in Medical Product Development to Support Labeling Claims.”
FitzSimmons, S. (1997). “High Dose Pancreatic Enzyme supplements and fibrosing colonopathy in children with cystic fibrosis.” Massachusetts Medical Society 336(18): 6.
Gass, J., M. T. Bethune, et al. (2007). “Combination enzyme therapy for gastric digestion of dietary gluten in patients with celiac sprue.” Gastroenterology 133(2): 472-80.
Gass, J., H. Vora, et al. (2006). “Effect of barley endoprotease EP-B2 on gluten digestion in the intact rat.” J Pharmacol Exp Ther 318(3): 1178-86.
Glade, M. J., D. Kendra, et al. (2001). “Improvement in protein utilization in nursing-home patients on tube feeding supplemented with an enzyme product derived from Aspergillus niger and Bromelain.” Nutrition 17(4): 348-350.
Green, P. H. (2007). “Where are all those patients with Celiac disease?” Am J Gastroenterol 102(7): 1461-3.
Green, P. H. and C. Cellier (2007). “Celiac disease.” N Engl J Med 357(17): 1731-43.
Hadjivassiliou, M. (2006). “Autoantibody targeting of brain and intestinal transglutaminase in gluten ataxia.” Department of Neurology and Gastroenterology 66: 373-377.
Hallert, C. (1998). “Quality of Life of Adult Coeliac Patients Treated for 10 Years.” Scand J Gastroenterol.
Hamilton, J. (1972). “Childhood celiac disease: response of treated patients to a small uniform daily dose of wheat gluten.” J Pediatrics(81): 885-93.
Hausch, F., L. Shan, et al. (2002). “Intestinal digestive resistance of immunodominant gliadin peptides.” Am J Physiol Gastrointest Liver Physiol 283(4): G996-G1003.
Hauser, W. (2007). “Development and Validation of the Celiac Disease Questionnaire (CDQ), a Disease-specific Health-related Quality of Life Measure for Adult Patients With Celiac Disease.” J Clin Gastroenterol 41: 157-166.
Hauser, W. (2007). “Predictors of Irritable Bowel-Type Symptoms and Healthcare-Seeking Behavior Among Adults With Celiac Disease.” Psychosomatic Medicine 69: 370-376.
Hegde, V. L. and Y. P. Venkatesh (2004). “Anaphylaxis to excipient mannitol: evidence for an immunoglobulin E-mediated mechanism.” Clinical & Experimental Allergy 34(10): 1602-1609.
Hill I D, D. M., Liptak G S, Colletti R B, Fasano A, Guandalini S, Hoffenberg E J, Horvath K, Murray J A, Pivor M, Seidman E G; (2005). “Guideline for the Diagnosis and Treatment of Celiac Disease in Children: Recommendations of the North American Society for Pediatric Gastroenterology, Hepatology and Nutrition . . . ” North American Society for Pediatric Gastroenterology, Hepatology and Nutrition. 40(1): 1-19.
Hischenhuber, C. (2006). “Allergen management in the food industry--potential and limitations.” Mol Nutr Food Res 49(1): 4-5.
Hogberg, L., L. Stenhammar, et al. (1997). “Anti-endomysium and anti-gliadin antibodies as serological markers for a very late mucosal relapse in a coeliac girl.” Acta Paediatrica 86(3): 335-336.
Holm, K., M. Mäki, et al. (1992). “Intraepithelial [gamma][delta] T-cell-receptor lymphocytes and genetic susceptibility to coeliac disease.” The Lancet 339(8808): 1500-1503.
Holm, K., M. Mäki, et al. (2006). “Oats in the treatment of childhood coeliac disease: a 2-year controlled trial and a long-term clinical follow-up study.” Alimentary Pharmacology & Therapeutics 23(10): 1463-1472.
Holm, K. H. (1993). “Correlation of HLA-DR alleles to jejunal mucosal morphology in healthy first-degree relatives of coeliac disease patients.” European Journal of Gastroenterology & Hepatology 5(1): 35-40.
Iltanen, Holm, et al. (1999). “Changing jejunal [gamma][delta] T cell receptor (TCR)-bearing intraepithelial lymphocyte density in coeliac disease.” Clinical & Experimental Immunology 117(1): 51-55.
Jarvinen, T. T. (2004). “Villous Tip Intraepithelial Lymphocytes as Markers of Early-Stage Coeliac Disease.” Scand J Gastroenterol 5.
Jarvinen, T. T., K. Kaukinen, et al. (2003). “Intraepithelial lymphocytes in celiac disease.” The American Journal of Gastroenterology 98(6): 1332-1337.
Kagnoff, M. F. (2007). “Celiac disease: pathogenesis of a model immunogenetic disease.” J Clin Invest 117(1): 41-9.
Kane, S. and M. J. Goldberg (2000). “Use of bromelain for mild ulcerative colitis.” Ann Intern Med 132(8): 680.
Kaukinen, K. (2000). “Intolerance to Cereals Is Not Specific for Coeliac Disease.” Scand J Gastroenterol 9.
Kaukinen, K. (2001). “Celiac Disease Without Villous Atrophy Revision of Criteria Called for.” Digestive Diseases and Sciences 46(4): 879-887.
Kaukinen, K. (2005). “Immunohistochemical Features in Antiendomysium Positive Patients with Normal Villous Architecture.” American Journal of Gastroenterology: 675-676.
Kaukinen, K. (2005). “Small-bowel muscosal transglutaminase 2-specific IgA deposits in coeliac disease without villous atrophy: A prospective and randomized clinical study.” Scand J Gastroenterol 40: 564-572.
Kaukinen, K., P. Collin, et al. (1998). “Small-Bowel Mucosal Inflammation in Reticulin or Gliadin Antibody-Positive Patients without Villous Atrophy.” Scandinavian Journal of Gastroenterology 33: 944-949.
Kaukinen, K., P. Collin, et al. (1999). “Wheat Starch-Containing Gluten-Free Flour Products in the Treatment of Coeliac Disease and Dermatitis Herpetiformis: A Long-Term Follow-up Study.” Scandinavian Journal of Gastroenterology 34: 163-169.
Kaukinen, K., L. Halme, et al. (2002). “Celiac disease in patients with severe liver disease: gluten-free diet may reverse hepatic failure.” Gastroenterology 122(4): 881-888.
Khosla, C., G. M. Gray, et al. (2005). “Putative efficacy and dosage of prolyl endopeptidase for digesting and detoxifying gliadin peptides.” Gastroenterology 129(4): 1362-3; author reply 1363.
Korponay-Szabo (2004). “In vivo targeting of intestinal and extraintestinal transglutaminase 2 by coeliac autoantibodies.” Gut 53: 641-48.
Korponay-Szabo, I. R., T. Raivio, et al. (2005). “Coeliac disease case finding and diet monitoring by point-of-care testing.” Aliment Pharmacol Ther 22(8): 729-37.
Kuitunen P, K. I., Savialhti E. (1982). “Morphometric study of the jejunal mucosa in various childhood enteropathies with special reference to intraepithelial lymphocytes.” J Pediatr Gastroenterol Nutr 1: 525-31.
Kurppa, K. (2009). “Diagnosing Mild Enteropathy Celiac Disease: A Randomized, Controlled Clinical Study.” Gastroentérologie Clinique et Biologique 136: 816-823.
Lee, A. and J. M. Newman (2003). “Celiac diet: Its impact on quality of life.” Journal of the American Dietetic Association 103(11): 1533-1535.
Lohiniemi, S. (1998). “Measuring quality of life in coeliac disease patients.” Changing Features of Coeliac Disease. The Finnish Coeliac Society. (Tampere 1998): 73-77.
Lopez, M. and L. Edens (2005). “Effective prevention of chill-haze in beer using an acid proline-specific endoprotease from Aspergillus niger.” J Agric Food Chem 53(20): 7944-9.
Macdonald, W. C., L. L. Brandborg, et al. (1964). “Studies of Celiac Sprue. Iv. The Response of the Whole Length of the Small Bowel to a Gluten-Free Diet.” Gastroenterology 47: 573-89.
Mäki, M. (1995). “The humoral immune system in coeliac disease.” Baillieres Clin Gastroenterol 9(2): 231-49.
Mäki, M. (1991). “Increase in gamma/delta T cell receptor bearing lymphocytes in normal small bowel mucosa in latent coeliac disease.” Gut 32: 1412-4.
Mäki, M. and P. Collin (1997). “Coeliac disease.” The Lancet 349(9067): 1755-1759.
Mäki, M., O. Hallstrom, et al. (1984). “Evaluation of a serum IgA-class reticulin antibody test for the detection of childhood celiac disease.” J Pediatr 105(6): 901-5.
Mäki, M., M. L. Landeaho, et al. (1989). “Postpubertal gluten challenge in coeliac disease.” Arch Dis Child 64: 1604-7.
Mäki, M., K. Mustalahti, et al. (2003). “Prevalence of Celiac Disease among Children in Finland.” N Engl J Med 348(25): 2517-2524.
Marsh, M. N. and P. T. Crowe (1995). “5 Morphology of the mucosal lesion in gluten sensitivity.” Bailliere's Clinical Gastroenterology 9(2): 273-293.
Marti, T., O. Molberg, et al. (2005). “Prolyl endopeptidase-mediated destruction of T cell epitopes in whole gluten: chemical and immunological characterization.” J Pharmacol Exp Ther 312(1): 19-26.
McHorney, C. A., J. E. Ware, Jr., et al. (1994). “The MOS 36-item Short-Form Health Survey (SF-36): III. Tests of data quality, scaling assumptions, and reliability across diverse patient groups.” Med Care 32(1): 40-66.
Mustalahti, K. (2002). “GlutenFree Diet and Quality of Life in Patients with Screen-Detected Celiac Disease.” Eff Clin Pract.
Mustalahti, K., S. Lohiniemi, et al. (2002). “Gluten-free diet and quality of life in patients with screen-detected celiac disease.” Eff Clin Pract 5(3): 105-13.
O'Leary, C. (2004). “Celiac Disease and the Transition from Childhood to Adulthood: A 28-Year Follow-Up.” American Journal of Gastroenterology.
Pasricha, P. J., D. E. Fleischer, et al. (1994). “Endoscopic perforations of the upper digestive tract: a review of their pathogenesis, prevention, and management.” Gastroenterology 106(3): 787-802.
Paterson, B. M., K. M. Lammers, et al. (2007). “The safety, tolerance, pharmacokinetic and pharmacodynamic effects of single doses of AT-1001 in coeliac disease subjects: a proof of concept study.” Aliment Pharmacol Ther 26(5): 757-66.
Peraaho, M., K. Kaukinen, et al. (2003). “Wheat-starch-based gluten-free products in the treatment of newly detected coeliac disease: prospective and randomized study.” Alimentary Pharmacology & Therapeutics 17(4): 587-594.
Peters, U., J. Askling, et al. (2003). “Causes of death in patients with celiac disease in a population-based Swedish cohort.” Arch Intern Med 163(13): 1566-72.
Piper, J. L., G. M. Gray, et al. (2004). “Effect of prolyl endopeptidase on digestive-resistant gliadin peptides in vivo.” J Pharmacol Exp Ther 311(1): 213-9.
Pyle, G. G., B. Paaso, et al. (2005). “Effect of pretreatment of food gluten with prolyl endopeptidase on gluten-induced malabsorption in celiac sprue.” Clin Gastroenterol Hepatol 3(7): 687-94.
Quine, M. A., G. D. Bell, et al. (1995). “Prospective audit of upper gastrointestinal endoscopy in two regions of England: safety, staffing, and sedation methods.” Gut 36(3): 462-467.
Raivio, T. (2006). “Self transglutaminase-based rapid coeliac disease antibody detection by a lateral flow method.” Aliment Pharmacol Ther 24: 147-154.
Reunala, T. L. (2001). “Dermatitis herpetiformis.” Clinics in Dermatology 19(6): 728-736.
Rizzello, C. G., M. De Angelis, et al. (2007). “Highly efficient gluten degradation by lactobacilli and fungal proteases during food processing: new perspectives for celiac disease.” Appl Environ Microbiol 73(14): 4499-507.
Rostom, A., J. A. Murray, et al. (2006). “American Gastroenterological Association (AGA) Institute Technical Review on the Diagnosis and Management of Celiac Disease.” Gastroenterology 131(6): 1981-2002.
Rubio-tapia, et al. (2010) Am J Gastroenterol 105, 1412-1420.
Salmi, T. T. (2006). “Endomysial antibody-negative coeliac disease: clinical characteristics and intestinal autoantibody deposits.” Gut 55(1746-1753).
Salmi, T. T. (2006). “Immunoglobulin A autoantibodies against transglutaminase 2 in the small intestinal mucosa predict forthcoming coeliax disease.” Aliment Pharmacol Ther 24(541-552).
Sanderson (1975). “Failure of laboratory and radiological studies to predict jejunal mucosal atrophy . . . ” Arch Dis Child 50: 526-31.
Shan, L., T. Marti, et al. (2004). “Comparative biochemical analysis of three bacterial prolyl endopeptidases: implications for coeliac sprue.” Biochem J 383(Pt 2): 311-8.
Shan, L., O. Molberg, et al. (2002). “Structural basis for gluten intolerance in celiac sprue.” Science 297(5590): 2275-9.
Shan, L., S. W. Qiao, et al. (2005). “Identification and analysis of multivalent proteolytically resistant peptides from gluten: implications for celiac sprue.” J Proteome Res 4(5): 1732-41.
Shiner (1973). “Ultrastructural changes suggestive of immune reactions in the jejunal mucosa of coeliac children following gluten challenge.” Gut 14 1-12
Shiner, M. and J. Ballard (1972). “Antigen-antibody reactions in jejunal mucosa in childhood coeliac disease after gluten challenge.” Lancet 1(7762): 1202-5.
Shiner, M. J. (1972). “Antigen-antibody reactions in jejunal mucosa in childhood coeliac disease after gluten challenge.” Lancet 1202-05.
Siegel, M., M. T. Bethune, et al. (2006). “Rational design of combination enzyme therapy for celiac sprue.” Chem Biol 13(6): 649-58.
Sollid, L. M. and C. Khosla (2005). “Future therapeutic options for celiac disease.” Nat Clin Pract Gastroenterol Hepatol 2(3): 140-7.
Stamnaes, J., B. Fleckenstein, et al. (2008). “The propensity for deamidation and transamidation of peptides by transglutaminase 2 is dependent on substrate affinity and reaction conditions.” Biochimica et Biophysica Acta (BBA)—Proteins & Proteomics 1784 (11): 1804-1811.
Stepniak, D., L. Spaenij-Dekking, et al. (2006). “Highly efficient gluten degradation with a newly identified prolyl endoprotease: implications for celiac disease.” Am J Physiol Gastrointest Liver Physiol 291(4): G621-9.
Stern, M. (2000). “Comparative Evaluation of Serologic Tests for Celiac Disease: A European Initiative Toward Standardization.” Journal of Pediatric Gastroenterology and Nutrition 31: 513-519.
Sturgess, R., P. Day, et al. (1994). “Wheat peptide challenge in coeliac disease.” The Lancet 343(8900): 758-761.
Sulkanen, S., T. Halttunen, et al. (1998). “Tissue transglutaminase autoantibody enzyme-linked immunosorbent assay in detecting celiac disease.” Gastroenterology 115(6): 1322-1328.
Svedlund, J., I. Sjodin, et al. (1988). “GSRS—a clinical rating scale for gastrointestinal symptoms in patients with irritable bowel syndrome and peptic ulcer disease.” Dig Dis Sci 33(2): 129-34.
Van Heel, D. A. and J. West (2006). “Recent advances in coeliac disease.” Gut 55(7): 1037-46.
Viljamaa, M., P. Collin, et al. (2005). “Is coeliac disease screening in risk groups justified? A fourteen-year follow-up with special focus on compliance and quality of life.” Alimentary Pharmacology & Therapeutics 22(4): 317-324.
Walker-Smith, J. (1990). “Revised criteria for diagnosis of coeliac disease . . . ” Arch Dis Child 65: 909-11.
West, J. (2003). “Seroprevalence, correlates, and characteristics of undetected coeliac disease in England.” Gut 2003(52): 960-965.

All references cited throughout this disclosure are expressly incorporated by reference herein.

We claim:

1. A method for protecting a patient from a deleterious effect of gluten ingestion, said method comprising administering orally to said patient a unit dose of ALV003 in an amount ranging from 150 mg to 3 g per day sufficient to prevent said deleterious effect,
wherein said unit dose is formulated as a combination of three stickpacks, one containing ALV001 or ALV001*, one containing ALV002, and one containing a flavor agent.

2. The method of claim 1, wherein the unit dose comprises as excipients cysteine and sodium metabisulfite.

3. The method of claim 2, wherein cysteine is present at a concentration of about 100 mg.

4. The method of claim 3, wherein sodium metabisulfite is present at a concentration of about 8 mg.

5. The method of claim 4, wherein a portion of the excipient is provided in the flavor-containing stickpack.

6. The method of claim 1, wherein separate components are admixed by dissolving in a potable liquid.

7. The unit dosage method of claim 6, wherein said potable liquid is water or a fruit juice.

8. The method of claim 1, wherein said patient has celiac disease, and said deleterious effect is intestinal mucosal injury.

9. The method of claim 1, wherein said administering occurs at mealtime.

10. The method of claim 1, wherein said dose is administered three times per day.

11. The method of claim 1, wherein the ALV003 has equal amounts of ALV001 and/or ALV001* and ALV002, wherein said ALV001 and/or ALV001* has a specific activity of at least 5000 or more proteolytic activity units per mg, and said ALV002 has a specific activity of at least 3000 or more proteolytic activity units per mg.

12. A unit dosage form of ALV003 for use in the method of claim 1.


SEQUENCE LISTING (4 sequences)

SEQ ID NO: 1 - protein, 401 aa, Artificial Sequence
  Synthetic polypeptide
MGSSHHHHHHSSGLVPRGSHMASMTGGQQMGRDPCSAIPMEDKDLESEEALWDLYERWQSAHRVRRHHAEKHRRFGTFKSNAHFIHSHNKRGDHPYRLHLNRFGDMDQAEFRATFVGDLRRDTPSKPPSVPGFMYAALNVSDLPPSVDWRQKGAVTGVKDQGKCGSCWAFSTVVSVEGINAIRTGSLVSLSEQELIDCDTADNDGCQGGLMDNAFEYIKNNGGLITEAAYPYRAARGTCNVARAAQNSPVVVHIDGHQDVPANSEEDLARAVANQPVSVAVEASGKAFMFYSEGVFTGECGTELDHGVAVVGYGVAEDGKAYWTVKNSWGPSWGEQGYIRVEKDSGASGGLCGIAMEASYPVKTYSKPKPTPRRALGARESLNSSSVDKLAAALEHHHHHH

SEQ ID NO: 2 - protein, 262 aa, Artificial Sequence
  Synthetic polypeptide
VSDLPPSVDWRQKGAVTGVKDQGKCGSCWAFSTVVSVEGINAIRTGSLVSLSEQELIDCDTADNDGCQGGLMDNAFEYIKNNGGLITEAAYPYRAARGTCNVARAAQNSPVVVHIDGHQDVPANSEEDLARAVANQPVSVAVEASGKAFMFYSEGVFTGECGTELDHGVAVVGYGVAEDGKAYWTVKNSWGPSWGEQGYIRVEKDSGASGGLCGIAMEASYPVKTYSKPKPTPRRALGARESLNSSSVDKLAAALEHHHHHH

SEQ ID NO: 3 - protein, 741 aa, Artificial Sequence
  Synthetic Polypeptide
MVSSHHHHHHSSGLVPRGSKNRLWLAMAAPLALATPVAFAQTPPTLAKDQAMPSLPPYPASPQVPLVEDHFGEKVSDPWRWLEADVRTDAKVAAWVQAQSAYTAAYLKQLPERAALEKRMKALIDYERFGLPQRRGASVFYSWNSGLMNQSQLLVRPADAPVGTKGRVLLDPNTWAKDGATALDAWAASDDGRLLAYSVQDGGSDWRTVKFVGVADGKPLADELKWVKFSGLAWLGNDALLYSRFAEPKEGQAFQALNYNQTVWLHRLGTPQSADQPVFATPELPKRGHGASVSSDGRWVVITSSEGTDPVNTVHVARVTNGKIGPVTALIPDLKAQWDFVDGVGDQLWFVSGDGAPLKKIVRVDLSGSTPRFDTVVPESKDNLESVGIAGNRLFASYIHDAKSQVLAFDLDGKPAGAVSLPGIGSASGLSGRPGDRHAYLSFSSFTQPATVLALDPATAKTTPWEPVHLTFDPADFRVEQVFYPSKDGTKVPMFIVRRKDAKGPLPTLLYGYGGFNVALTPWFSAGFMTWIDSGGAFALANLRGGGEYGDAWHDAGRRDKKQNVFDDFIAAGEWLIANGVTPRHGLAIEGGSNGGLLIGAVTNQRPDLFAAASPAVGVMDMLRFDQFTAGRYWVDDYGYPEKEADWRVLRRYSPYHNVRSGVDYPAILVTTADTDDRVVPGHSLKYTAALQTAAIGPKPHLIRIETRAGHGSGKPIDKQIEETADVQAFLAHFTGLTPRP

SEQ ID NO: 4 - protein, 6 aa, Artificial Sequence
  Synthetic 6xHIS tag sequence
HHHHHH